SEQUENCE LISTING (6 sequences)

SEQ ID NO: 1 - DNA, 1422 bp, Artificial Sequence
  source 1..1422: /organism=“Artificial Sequence” /note=“Optimized endocellulase Cel5A gene sequence from Bacillus subtilis” /mol_type=“unassigned DNA”
ctcgaggcaggcaccaaaacaccggttgcaaaaaatggtcagctgagtattaaaggcacccagctggttaatcgtgatggtaaagcagtgcagctgaaaggtattagcagccatggtctgcagtggtatggtgaatttgttaataaagacagcctgaaatggctgcgtgatgattggggtattaccgtttttcgtgcagcaatgtataccgcagatggtggctatattgataatccgagcgttaaaaacaaagtgaaagaagcagttgaagcagccaaagaactgggcatttatgtgattattgattggcacattctgaacgatggtaatccgaatcagaacaaagaaaaagcgaaagaatttttcaaagaaatgagcagcctgtatggcaataccccgaatgtgatttatgaaattgccaatgaaccgaatggtgatgtgaattggaaacgcgatatcaaaccgtatgccgaagaagtgattagcgttattcgtaaaaatgacccggacaacattattatcgttggcaccggcacctggtcacaggatgttaatgatgcagcagatgatcaactgaaagatgccaatgttatgtatgccctgcatttttatgcaggcacccatggtcagtttctgcgtgataaagcaaattatgcactgagcaaaggtgcaccggtttttgttaccgaatggggcaccagtgatgcaagcggtaatggtggtgtttttctggatcagagccgtgaatggctgaattatctggatagcaaaaccattagctgggtgaactggaatctgagcgataaacaagaaagcagcagcgcactgaaaccgggtgcaagcaaaacaggtggttggcgtctgagtgatctgagcgcaagcggcacctttgttcgtgaaaatattctgggtacaaaagatagcaccaaagacattccggaaacaccggcaaaagataaaccgacccaagaaaatggtattagcgttcagtatcgtgccggtgatggtagcatgaatagcaatcagattcgtccgcagctgcagatcaaaaacaatggtaataccaccgtggatctgaaagacgttaccgcacgttattggtataacgccaaaaacaaaggccagaatgtggattgtgattatgcacagctgggttgtggtaatatgacccataaatttgtgacactgcacaaaccgaaacagggtgcagatacctatctggaactgggtttcaaaaatggcaccctggcaccgggtgcctcaaccggtaatattcagctgcgtctgcataacgatgattggagcaattatgcccagagcggtgattatagctttttcaaaagcaacacctttaaaaccaccaaaaaaatcaccctgtatgaccagggtaaactgatttggggcaccgaaccgaacggtacc

SEQ ID NO: 2 - protein, 474 aa, Artificial Sequence
  From Seq_1 deduced endocellulase Cel5A peptide sequence
LEAGTKTPVAKNGQLSIKGTQLVNRDGKAVQLKGISSHGLQWYGEFVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPVFVTEWGTSDASGNGGVFLDQSREWLNYLDSKTISWVNWNLSDKQESSSALKPGASKTGGWRLSDLSASGTFVRENILGTKDSTKDIPETPAKDKPTQENGISVQYRAGDGSMNSNQIRPQLQIKNNGNTTVDLKDVTARYWYNAKNKGQNVDCDYAQLGCGNMTHKFVTLHKPKQGADTYLELGFKNGTLAPGASTGNIQLRLHNDDWSNYAQSGDYSFFKSNTFKTTKKITLYDQGKLIWGTEPNGT

SEQ ID NO: 3 - DNA, 2382 bp, Artificial Sequence
  source 1..2382: /organism=“Artificial Sequence” /note=“Optimized gene sequence of an exocellulase CelK from Clostridium thermocellum” /mol_type=“unassigned DNA”
ctcgagctggaagataaaagcagcaaactgccggattacaaaaacgatctgctgtatgaacgtacctttgatgaaggtctgtgttttccgtggcatacctgtgaagatagcggtggtaaatgtgattttgccgttgttgatgttccgggtgaaccgggtaataaagcatttcgtctgaccgttattgataaaggccagaataaatggtcagtgcagatgcgtcatcgtggtattaccctggaacagggtcatacctataccgttcgttttaccatttggagcgataaaagctgtcgtgtgtatgcaaaaattggtcagatgggcgaaccgtataccgaatattggaataataactggaacccgtttaatctgacccctggtcagaaactgaccgttgaacagaattttaccatgaactatccgaccgatgatacctgcgaattcacctttcatctgggtggtgaactggcagcaggcaccccgtattatgtttatctggatgatgttagcctgtacgatccgcgttttgttaaaccggttgaatatgttctgccgcagccggatgttcgtgttaatcaggttggttatctgccgtttgccaaaaaatacgcaaccgttgttagcagcagcaccagtccgctgaaatggcagctgctgaatagcgcaaatcaggtggttctggaaggcaataccattccgaaaggtctggataaagatagccaggattatgtgcattggatcgatttcagcaactttaaaaccgaaggcaaaggctactactttaaactgccgaccgttaatagcgataccaattatagccatccgtttgatattagcgcagatatctatagcaaaatgaaatttgatgccctggccttcttttatcataaacgtagcggtattccgatcgaaatgccgtatgcaggcggtgaacagtggacccgtccggcaggtcatattggtattgaaccgaataaaggtgataccaatgttccgacctggcctcaggatgatgaatatgcaggtcgtccgcagaaatattacaccaaagatgttaccggtggttggtatgatgccggtgatcatggcaaatatgttgtgaatggtggtattgcagtttggaccctgatgaacatgtatgagcgtgccaaaattcgcggtattgccaatcagggtgcatataaagatggtggcatgaatattccggaacgcaataatggttatccggacattctggatgaagcacgttgggaaattgaatttttcaaaaaaatgcaggttaccgaaaaagaagatccgagcattgcaggtatggtgcatcataaaattcatgattttcgttggaccgcactgggtatgctgccgcatgaagatccgcagcctcgttatctgcgtccggttagcaccgcagcaaccctgaattttgcagccaccctggcacagagcgcacgtctgtggaaagattatgatccgacctttgcagcagattgtctggaaaaagcagaaattgcatggcaggcagcactgaaacatccggatatttatgcagaatatacaccgggtagtggtggtccgggtggcggtccgtataatgatgattatgttggtgatgagttttattgggcagcctgtgaactgtatgttaccaccggcaaagatgagtataaaaactatctgatgaactcaccgcactacctggaaatgcctgcaaaaatgggtgaaaatggtggtgcaaatggtgaagataatggtctgtggggttgttttacctggggtacaacccagggtctgggcaccattacactggcactggtggaaaatggtctgcctgcaaccgatattcagaaagcacgtaataacattgcaaaagcagccgatcgttggctggaaaatattgaagaacagggttatcgtctgccgattaaacaggccgaagatgaacgtggtggctatccgtggggtagcaatagctttattctgaatcagatgatcgtgatgggctatgcctatgattttaccggcaatagcaaatatctggacggtatgcaggatggtatgagctatctgctgggtcgcaatggtctggatcagagctatgtgaccggttatggtgaacgtccgctgcagaatccgcatgatcgcttttggacaccgcagaccagcaaaaaattccctgcaccgcctccgggtattattgcgggtggtccgaatagccgttttgaagatcctaccattaccgcagcagtgaaaaaagatacccctcctcagaaatgctatatcgatcataccgatagctggtccaccaatgaaattaccgttaattggaatgcaccgtttgcatgggttaccgcgtacctggatgaaattgatctgattggtacc

SEQ ID NO: 4 - protein, 794 aa, Artificial Sequence
  From Seq_3 deduced peptide sequence from exocellulase CelK
LELEDKSSKLPDYKNDLLYERTFDEGLCFPWHTCEDSGGKCDFAVVDVPGEPGNKAFRLTVIDKGQNKWSVQMRHRGITLEQGHTYTVRFTIWSDKSCRVYAKIGQMGEPYTEYWNNNWNPFNLTPGQKLTVEQNFTMNYPTDDTCEFTFHLGGELAAGTPYYVYLDDVSLYDPRFVKPVEYVLPQPDVRVNQVGYLPFAKKYATVVSSSTSPLKWQLLNSANQVVLEGNTIPKGLDKDSQDYVHWIDFSNFKTEGKGYYFKLPTVNSDTNYSHPFDISADIYSKMKFDALAFFYHKRGSIPIEMPYAGGEQWTRPAGHIGIEPNKGDTNVPTWPQDDEYAGRPQKYYTKDVTGGWYDAGDHGKYVVNGGIAVWTLMNMYERAKIRGIANQGAYKDGGMNIPERNNGYPDILDEARWEIEFFKKMQVTEKEDPSIAGMVHHKIHDFRWTALGMLPHEDPQPRYLRPVSTAATLNFAATLAQSARLWKDYDPTFAADCLEKAEIAWQAALKHPDIYAEYTPGSGGPGGGPYNDDYVGDEFYWAACELYVTTGKDEYKNYLMNSPHYLEMPAKMGENGGANGEDNGLWGCFTWGTTQGLGTITLALVENGLPATDIQKARNNIAKAADRWLENIEEQGYRLPIKQAEDERGGYPWGSNSFILNQMIVMGYAYDFTGNSKYLDGMQDGMSYLLGRNGLDQSYVTGYGERPLQNPHDRFWTPQTSKKFPAPPPGIIAGGPNSRFEDPTITAAVKKDTPPQKCYIDHTDSWSTNEITVNWNAPFAWVTAYLDEIDLIGT

SEQ ID NO: 5 - DNA, 1353 bp, Artificial Sequence
  source 1..1353: /organism=“Artificial Sequence” /note=“Optimized gene sequenze of beta-glucosidase BglA from Clostridium thermocellum” /mol_type=“unassigned DNA”
ctcgagagcaaaattaccttcccgaaagattttatctggggtagcgcaaccgcagcatatcagattgaaggtgcatataatgaagatggcaaaggcgaaagcatttgggatcgttttagccatacaccgggtaatattgcagatggtcataccggtgatgttgcctgtgatcattatcatcgttatgaagaggatatcaaaatcatgaaagagatcggcatcaaaagctatcgctttagcattagctggcctcgtatttttccggaaggcaccggtaaactgaatcagaaaggtctggatttctataaacgtctgaccaatctgctgctggaaaatggtattatgcctgcaattaccctgtatcattgggatctgccgcagaaactgcaggataaaggtggttggaaaaatcgtgataccaccgattatttcaccgaatatagcgaggtgatctttaaaaacctgggtgatattgtgccgatctggtttacccataatgaaccgggtgttgttagcctgctgggtcattttctgggtattcatgcaccgggtattaaagatctgcgtaccagcctggaagttagccataacctgctgctgagccatggtaaagcagttaaactgtttcgcgagatgaatattgatgcccagattggtattgcactgaacctgagctatcattatccggcaagcgaaaaagcagaagatattgaagcagcagaactgagctttagcctggcaggtcgttggtatctggatccggttctgaaaggtcgttatccggaaaatgcactgaaactgtacaaaaaaaaaggcatcgaactgtcgttcccggaagatgatctgaaactgattagccagccgattgattttatcgcctttaacaattatagcagcgagttcatcaaatatgatccgagcagcgaaagcggttttagtccggcaaatagcattctggaaaaattcgagaaaaccgatatgggctggattatctatccggaaggtctgtatgatctgctgatgctgctggatcgtgattatggcaaaccgaatattgtgattagcgaaaatggtgcagccttcaaagatgaaattggtagcaatggcaaaatcgaggataccaaacgtatccagtatctgaaagattatctgacccaggcacatcgtgcaattcaggatggtgttaatctgaaagcatattatctgtggtccctgctggataattttgaatgggcatatggttacaacaaacgctttggtattgtgcacgtgaattttgataccctggaacgcaaaatcaaagatagcggctattggtataaagaggtgatcaaaaacaacggctttggtacc

SEQ ID NO: 6 - protein, 451 aa, Artificial Sequence
  From Seq_5 deduced peptide sequence of beta-glucosidase BglA
LESKITFPKDFIWGSATAAYQIEGAYNEDGKGESIWDRFSHTPGNIADGHTGDVACDHYHRYEEDIKIMKEIGIKSYRFSISWPRIFPEGTGKLNQKGLDFYKRLTNLLLENGIMPAITLYHWDLPQKLQDKGGWKNRDTTDYFTEYSEVIFKNLGDIVPIWFTHNEPGVVSLLGHFLGIHAPGIKDLRTSLEVSHNLLLSHGKAVKLFREMNIDAQIGIALNLSYHYPASEKAEDIEAAELSFSLAGRWYLDPVLKGRYPENALKLYKKKGIELSFPEDDLKLISQPIDFIAFNNYSSEFIKYDPSSESGFSPANSILEKFEKTDMGWIIYPEGLYDLLMLLDRDYGKPNIVISENGAAFKDEIGSNGKIEDTKRIQYLKDYLTQAHRAIQDGVNLKAYYLWSLLDNFEWAYGYNKRFGIVHVNFDTLERKIKDSGYWYKEVIKNNGFGT

(19) United States
(12) Patent Application Publication
(10) Pub. No.: US 2014/0377811 A1
(43) Pub. Date: Dec. 25, 2014

(54) WHOLE-CELL BIOCATALYSTS IN THE DEGRADATION OF CELLULOSIC BIOMASS

(71) Applicant: ZYRUS Beteiligungsgesellschaft mbH & Co. Patente I KG, Schonefeld/Waltersdorf (DE)

(72) Inventors: Michael Jurgens, Hannover (DE); Andreas Rohardt, Hermannsburg (DE); Ruth Maas, Dusseldorf (DE); Tatjana Brossette, Dusseldorf (DE)

(73) Assignee: ZYRUS Beteiligungsgesellschaft mbH & Co. Patente I KG, Schonefeld/Waltersdorf (DE)

(21) Appl. No.: 14/203,594

(22) Filed: Mar. 11, 2014

(30) Foreign Application Priority Data
Mar. 11, 2013 (EP) 13158634.9

(51) Int. Cl. C12P 19/14; C12P 19/02; C12P 19/12; C12N 9/42

(52) U.S. Cl. CPC C12P 19/14; C12N 9/2437; C12P 19/02; C12P 19/12
USPC 435/99; 536/23.2; 435/320.1; 435/209; 435/252.3; 435/471; 435/252.33

(57) ABSTRACT

The present invention concerns micro-organisms which present cellulases on their surface. Corresponding micro-organisms were produced with the aid of corresponding plasmids which encode a section comprising a signal peptide, a heterologous cellulase, an optional protease recognition site, a transmembrane linker and a transporter domain of an autotransporter or a variant thereof. Such micro-organisms were advantageously used in the conversion of cellulose into cellobiose and/or glucose. It was also possible to recover the micro-organisms from the reaction mixture following conversion from simple substrates. Also, a combination of various micro-organisms, which were populated with exocellulases, endocellulases and beta-glucosidases, were used to produce glucose from cellulose or wood.

The invention concerns a nucleic acid molecule, comprising the following components:

(1) a section which encodes a signal peptide, (2) a section which encodes a heterologous cellulase, (3) an optional section which encodes a protease-recognition site, (4) a section which encodes a transmembrane linker, and (5) a section which encodes a transporter domain of an autotransporter or of a variant thereof.

Further aspects of the present invention concern polypeptides, which are encoded by a nucleic acid molecule as described above, micro-organisms, which present corresponding polypeptides on their surface, and membrane fractions obtainable from such micro-organisms. The present invention also concerns a method for the production of reaction products, in particular of cellulose reaction products, using the micro-organisms or membrane fractions, and also the use of micro-organisms and/or membrane preparations to produce products from a reaction which is catalysed by a cellulase.

Cellulases are enzymes which can degrade cellulose into its base unit, beta-glucose. Cellulases are only formed from a small part of the plant-degrading organisms, such as for example special bacteria and flagellates, and from wood-degrading micro-organisms, such as fungi. With the aid of these enzymes, the corresponding micro-organisms are capable of splitting and metabolically recycling cellulose, which normally also consists of 3000 to 15,000 glucose molecules, into degradation products and ultimately into glucose. Cellulases include, in particular, three enzyme types, whose combined effects enable a rational digestion of the cellulose molecules:

1. Endocellulases (enzyme class EC 3.2.1.4): These enzymes split cellulose into larger sections and, unlike other cellulases, can also work within cellulose chains. In particular, endocellulases can become active in “amorphous regions”, in which the cellulose molecules are disordered in relation to each other. As a result of this, the enzymes generate a larger number of chain ends.

2. Exocellulases (EC 3.2.1.91): These enzymes only split cellulose off from its ends, while continuously shortening the cellulose chains. The reaction of the exocellulases typically cause cellobiose units (disaccharides), but also glucose, cellotriose or cellutetraose to split off from the cellulose chain.

3. Cellobiase or beta-glucosidase (EC 3.2.1.21): This group of enzymes splits the beta-glycosidic bond between the two glucose molecules of the cellobiose and thereby makes glucose available for further metabolic processes.

The three enzyme types mentioned are each capable of attacking cellulosic biomass on their own. However, it is assumed that a combination of said enzyme types behaves synergistically to a high degree as they attack different natural structural units of the cellulosic biomass. In particular, when a cellobioase is used, it can be expected that the degradation of the cellobioses, cellotrioses and other higher cellooligosaccharides provided by the endocellulases and exocellulases into glucose leads to a displacement of the equilibrium of the enzymatically catalysed reactions and so encourages a more effective conversion.

Cellulose and hemicellulose are main components of wood and many plants and so represent a freely-available raw material. One problem with the conversion of the material, however, lies in the difficulty of cleaving it, for example into glucose, which poses an obstacle to its extensive use in obtaining bioethanol. In the state of the art, there are at present some purified enzymes being used to decompose cellulose into glucose, which generally have to be isolated from fungal cultures such as Trichoderma reesei or C. acetobulyticum (cf. Shah et al., App. Biochem. Biotech. (1991), vol. 18/29, 99-106). The purification of the individual enzymes and the mixing of an effective enzyme cocktail prior to use in the decomposition of the cellulosic biomass represents a significant cost factor in today's state of the art. It is also impossible in the state of the art to recover the corresponding free enzymes from the product obtained. There is therefore a need for a ready-made form of cellulases, which, firstly, enables a simplified manufacture and mixing process and secondly allows the enzymes to be recovered after they have been used, in a simple way.

One problem of the present invention therefore lies in providing an agent with a cellulase activity, whereby the agent is not only easily accessible for substrate molecules, but can also be amplified, recycled and regenerated in a simple fashion. This should enable several catalysis steps, without the need to repeatedly manufacture new agent. A further problem of the present invention lies in providing an agent which exhibits favourable properties in respect of its genetics, thermostability, storability and stability.

The inventors have discovered, surprisingly, that by using an autotransporter system, cellulases can be expressed on the surface of micro-organisms and are sufficiently stable with respect to the reaction conditions for the splitting of cellulose and the later separation of the products obtained. It was also, surprisingly, found that micro-organisms which carry corresponding cellulases on their surface can be incubated and remain stable in various solutions and buffers under varying conditions over a long period.

The autodisplay represents an elegant tool for presenting recombinant proteins on the bacteria surface. This expression system is based on the secretion mechanism of the protein family of the autotransporters, which belong to the Type V secretion system.

In gram-negative bacteria, the autotransporter pathway was formed both for the transport of proteins to the cell surface as well as for the secretion of proteins in the extracellular space (Jose and Meyer, 2007). The autotransporter proteins are synthesised as precursor proteins, which meet all the structural requirements for the transport to the cell surface (Jose, 2006). They are synthesised with an N-terminal signal peptide, which is typical for the Sec-pathway which enables the crossing of the inner membrane. Once in the periplasm, the C-terminal part of the precursor, after splitting off the signal peptide, folds itself into the outer membrane as a pore-like structure known as a 6-barrel. The N-terminal attached passenger domain is translocated through this pore to the surface (Jose et al., 2002). There it can be split off—either autoproteolytically or by an additional protease—or remain anchored to the cell envelope via the transporter domain.

Replacing the natural passenger by a recombinant protein leads to the surface translocation thereof. To do so, genetic engineering processes must be used to construct an artificial precursor, consisting of a signal peptide, the recombinant passenger, the β-barrel and a linker region in between, which is necessary for unrestricted access to the surface. The AIDA-I autotransporter has already been successfully used in this way for an efficient surface display of various passenger domains (Henderson et al., 2004). In the autodisplay system, a self-association of sub-units on an active enzyme has been observed, for example in the dimeric enzyme sorbitol-dehydrogenase (Jose, 2002; Jose and von Schwichow, 2004).

In particular, the autodisplay technology is an expression method for specific proteins on the surface of the outer membrane of E. coli and other gram-negative bacteria, with the autodisplay system being based on the natural secretion mechanism of the autotransporter proteins (A. Banerjee et al. (2002)). In this case the transport of the recombinant passenger protein can take place simply by inserting its coding sequence in the reading frame, of its coding sequence between the signal peptide and the translocating domain of the autodisplay vector using ordinary genetic engineering techniques. The signal peptide can be obtained from a sub-unit of the cholera toxin, and it can be combined with an artificial promoter. Consequently, the passenger protein intended for translocation across the outer membrane is expressed as recombinant fusion protein with another protein, known as an autotransporter, on the outer membrane of E. coli (AIDA-I) (Jose, 2006). The C-terminal moiety of the autotransporter protein forms a pore-like structure (β-barrel) within the outer membrane of E. coli. membrane (Jose, 1995, 2006, 2007).

The concept of the autodisplay system, first described by Jose et al. (1995), has been known for more than 15 years. In the meantime, it has been shown that it could be used to produce cells with immobilised nitrilases thereon (cf. WO 2011/057820) and redox-active enzymes, which in turn regenerate redox factors, in particular NAD/NADH and NADP/NADPH (cf. WO 2012/025628), which displayed the desired activity. Nevertheless, transferring the autodisplay system to a new enzyme class still poses considerable challenges to the person skilled in the art, especially since a correct folding of the proteins, in the absence of the chaperones which are sometimes necessary, is not guaranteed. So far the state of the art has neither taught nor suggested the use of the autotransporter system for the immobilisation of cellulases.

The problem of the present invention is solved by the subject-matter of the independent claims. Preferred embodiments can be derived from the dependent claims.

The problem of the present invention is solved according to a first aspect by a nucleic acid molecule, comprising

(1) a section encoding a signal peptide,

(2) a section encoding a heterologous cellulase or a variant thereof,

(3) an optional section encoding a protease recognition site,

(4) a section encoding a transmembrane linker, and

(5) a section encoding a transporter domain of an autotransporter or a variant thereof.

Further aspects of the present invention concern proteins and polypeptides, obtainable by transcription of the corresponding nucleic acid molecules, and micro-organisms containing the corresponding proteins and membrane preparations obtainable from such micro-organisms. A further aspect of the present invention concerns a method for producing a reaction product using at least one cellulase, comprising the following steps:

(i) preparation of at least one micro-organism, which presents a cellulase on its surface, and/or a membrane preparation of this micro-organism, and

(ii) bringing the at least one micro-organism and/or membrane preparation into contact with one or more cellulase substrates under conditions compatible with cellulase activity.

Finally, the present invention also concerns the use of the micro-organisms or membrane preparations described to manufacture reaction products which can be produced from cellulase catalysis, and also methods for the production of corresponding micro-organisms and membrane preparations.

In one preferred embodiment of the present invention, the nucleic acid molecule is functionally linked with a sequence for expression regulation. In one preferred embodiment the term “sequence for expression regulation”, as used here, refers to a nucleic acid sequence which can regulate the level of expression of a nucleic acid molecule, preferably downstream from the sequence for expression regulation. The sequence for expression regulation can, for example, be a promoter. The person skilled in the art is familiar with sequences suitable for expression regulation and methods for the functional linking of these sequences with a nucleic acid molecule. In connection with the present invention, expression regulation sequences are especially preferred which code for expression regulators which can be activated with the aid of the inductors isopropylthioglucopyranoside (IPTG) or arabinose, in particular L-arabinose.

In one preferred embodiment of the present invention, the nucleic acid molecule is part of a recombinant plasmid.

In one preferred embodiment of the present invention, the term “heterologous”, as used here, refers to a nucleic acid molecule which has been constructed using genetic engineering methods, for example by splicing a sequence for expression regulation and a sequence to be expressed, which is not normally under the control of this sequence for expression regulation, or by using a sequence which has a point mutation from the original sequence. So if even just one section of a construct is described as heterologous, this therefore implies that the entire construct is heterologous. The person skilled in the art is familiar with genetic engineering methods. In a further preferred embodiment, the term “heterologous”, as used here, refers to a nucleic acid molecule encoding a polypeptide, which is spliced to a sequence for expression regulation, and/or a fused sequence, which is derived from a different organism than the sequence for expression regulation and is to be expressed. In a further preferred embodiment, the term “heterologous”, as used here in connection with a cellulase polypeptide, means that the section of the nucleic acid molecule which codes the cellulase has been obtained or taken from a different organism than at least one further section, such as the transporter domain or the sequence for expression regulation or the transmembrane linker. For example, the cellulase is heterologous if it has been obtained from Bacillus subtilis, but all other sequences were obtained from E. coli. In a further preferred embodiment, the term “heterologous”, as used here, means that the nucleic acid sequence described as heterologous has been derived from an organism which is different from the host or intended host used to express or to amplify this nucleic acid sequence.

In one especially preferred embodiment, the nucleic acid molecule comprises the sequences SEQ ID NO:1, SEQ ID NO:3 or SEQ ID NO:5 or variants thereof.

In one preferred embodiment of the present invention, the term “signal peptide”, as used here, refers to a sequence of amino acids, preferably at the N-terminus of a polypeptide, with the effect that the polypeptide, when it is expressed in the cytosol of a host cell, is translocated to a specific compartment of the cell, preferably a different compartment from the cytosol. In one especially preferred embodiment, the host cell is a gram-negative bacteria cell and the signal peptide causes the nascent or completed polypeptide to be translocated into the periplasm or the outer membrane of the gram-negative bacteria cell.

In a further preferred embodiment of the present invention, the term “protease-recognition site”, as used here, refers to a specific amino acid sequence pattern in a polypeptide, where this sequence pattern is specifically recognised by said protease, such that it binds to and cleaves the polypeptide.

Within the scope of the present invention, it is also preferred if the term “transmembrane linker”, as used here, refers to a flexible polypeptide section, which is used to link the autotransporter domain with the redox factor-regenerating polypeptide, but is flexible enough to enable an independent folding and/or transport of the redox factor-regenerating polypeptide.

In one preferred embodiment of the present invention, the term “transporter domain of an autotransporter” refers to a domain which can be used to the obtain the expression product of the nucleic acid molecule, if it is synthesized ribosomally inside the cell, preferably in the bacterial cytoplasm, and is translocated to the outer membrane of the cell, preferably to the side of the outer membrane that is exposed to the extracellular environment. In an especially preferred embodiment the transporter domain has the effect that said expression product is located on the external surface of the outer membrane. In a preferred embodiment of the present invention the transporter domain of an autotransporter is a protein that is located on the outer membrane of the cell, and the section encoding a cellulase is part of a domain, loop or some other part of the transporter domain or is fused thereto, so that the cellulase is presented on the surface of the cell. In a further preferred embodiment of the present invention, the transporter domain of an autotransporter is a protein of a system which can be used to present polypeptides on the surface of a cell.

In a preferred embodiment, variants of amino acids or nucleic acid sequences are referred to in the present application explicitly, for example by the name or the deposition number or even by the term “variant of”, or implicitly, for example by a description of their function in the context of the present invention. In a preferred embodiment the term “variant”, as used here, comprises amino acid or nucleic acid sequences that are 60, 70, 75, 80, 85, 90, 92, 94, 95, 96, 97, 98 or 99% identical to the amino acid serving as reference and, under comparable conditions, display similar cellulase activity. In one preferred embodiment, the term “variant” comprises, with respect to an amino acid sequence, those amino acid sequences that have one or more conservative amino acid exchanges relative to the reference sequence. In one preferred embodiment, the terms “variants” of an amino acid sequence or nucleic acid sequence comprise active sections and/or fragments of the amino acid sequence or nucleic acid sequence. In one preferred embodiment, the term “active section”, as used here, refers to an amino acid sequence or a nucleic acid sequence that is shorter than the amino acid or nucleic acid sequence of full length, but still retains at least some of its essential biological activity, e.g. as a cellulase. In one preferred embodiment the term “variant” of a nucleic acid comprises the nucleic acids of the complementary strand, which—preferably under stringent conditions—hybridise with the reference nucleic acid. The stringency of hybridisation reactions can readily be determined by the person skilled in the art and is usually an empirical calculation, as a function of probe length, wash temperature and salt concentration. In general, longer probes require higher temperatures for perfect annealing, while shorter probes require lower temperatures. Hybridisation generally depends on the capacity of the denatured DNA for renaturation, when complementary strands are in an environment that has a temperature below the melting point of the strands. The higher the degree of desired homology between the probe and a hybridisable sequence, the higher the relative temperature that can be applied. It therefore follows that higher relative temperatures tend to make the reaction conditions more stringent, whereas lower temperatures reduce this stringency. Further details and an explanation of the stringency of hybridisation reactions can be found in Ausubel et al. (1995). In another preferred embodiment the term “variant” of a nucleic acid or amino acid refers to a nucleic acid or amino acid that has, at least up to a certain degree, the same biological activity and/or function as the reference nucleic acid or amino acid. In one preferred embodiment, the term “variant” of a nucleic acid sequence, as used here, refers to another nucleic acid sequence, which encodes an amino acid sequence that is similar to the reference amino acid sequence.

In one preferred embodiment, the term “cellulase” refers to an enzyme from class EC 3.2.1.4, EC 3.2.1.91 or EC 3.2.1.21. In a further preferred embodiment, the cellulase is a cellulase from Bacillus subtilis or Clostridium thermocellum. The cellulase can be an endo-, exo- or exocellulase or a beta-glucosidase. Within the scope of the present invention it is, however, preferable, if it is an endocellulase from Bacillus subtilis, an exocellulase from Clostridium thermocellum or a beta-glucosidase from Clostridium thermocellum. It is especially preferable if it is an enzyme with a sequence according to SEQ ID NO:2, SEQ ID NO:4 or SEQ ID NO:6 or variant thereof.

Preferably, the cellulase is fused with a transporter domain of an autotransporter.

The transporter domain of the autotransporter according to the invention can be any transporter domain of an autotransporter and is preferably capable of forming a β-barrel structure. An extensive description of β-barrel structures and preferred examples for β-barrel autotransporters are disclosed in WO 97/35022 and form part of the present description by reference. Henderson et al. (2004) describe autotransporter proteins with suitable autotransporter domains (a summary can be found in Table 1 in Henderson et al., 2004). The disclosure of Henderson et al. (2004) forms part of the present description by reference. For example the transporter domain of the autotransporter can be selected from: Ssp (P09489, S. marcescens), Ssp-h1 (BAA33455, S. marcescens), Ssp-h2 (BAA11383, S. marcescens), PspA (BAA36466, P. fluorescens), PspB (BAA36467, P. fluorescens), Ssa1 (AAA80490. P. haemolytica), SphB1 (CAC44081, B. pertussis), AspA/NalP (AAN71715, N. meningitidis), VacA (Q48247, H. pylori), AIDA-I (Q03155, E. coli), IcsA (AAA26547, S. flexneri), MisL (AAD16954, S. enterica), TibA (AAD41751, E. coli), Ag43 (P39180. E. coli), ShdA (AAD25110. S. enterica), AutA (CAB89117, N. meningitidis), Tsh (154632, E. coli), SepA (CACO5786, S. flexneri), EspC (AAC44731, E. coli), EspP (CAA66144, E. coli), Pet (AAC26634, E. coli), Pic (AAD23953, E. coli), SigA (AAF67320. S. flexneri), Sat (AAG30168, E. coli), Vat (AA021903, E. coli), EpeA (AAL18821, E. coli), EatA (AA017297, E. coli), EspI (CAC39286, E. coli), EaaA (AAF63237, E. coli), EaaC (AAF63038, E. coli), Pertactin (P14283, B. pertussis), BrkA (AAA51646, B. pertussis), Tef (AAQ82668, B. pertussis), Vag8 (AAC31247, B. pertussis), PmpD (084818, C. trachomatis), Pmp20 (Q9Z812, C. pneumoniae), Pmp21 (Q9Z6U5, C. pneumoniae), IgA1 protease (NP_{—}283693, N. meningitidis), App (CAC14670. N. meningitidis), IgA1 protease (P45386, H. influenzae), Hap (P45387, H. influenzae), rOmpA (P15921, R. rickettsii), rOmpB (Q53047, R. rickettsii), ApeE (AAC38796, S. enterica), EstA (AAB61674, P. aeruginosa), Lip-1 (P40601, X. luminescens), McaP (AAP97134, M. catarrhalis), BabA (AAC38081, H. pylori), SabA (AAD06240. H. pylori), AlpA (CAB05386, H. pylori), Aae (AAP21063, A. actinomycetem-comitans), NanB (AAG35309, P. haemolytica) and variants of these autotransporters. For each of the examples of autotransporter proteins, examples of suitable GenBank accession numbers and species, from which the autotransporters can be obtained, are given in parentheses. Within the scope of the present invention, the transporter domain of the autotransporter is preferably selected from the group comprising Ssp, Ssp-h1, Ssp-h2, PspA, PspB, Ssa1, SphB1, AspA/NalP, VacA, AIDA-I, IcsA, MisL, TibA, Ag43, ShdA, AutA, Tsh, SepA, EspC, EspP, Pet, Pic, SigA, Sat, Vat, EpeA, EatA, EspI, EaaA, EaaC, Pertactin, BrkA, Tef, Vag8, PmpD, Pmp20. Pmp21, AgA1 protease, App, Hap, rOmpA, rOmpB, ApeE, EstA, Lip-1, McaP, BabA, SabA, AlpA, Aae, NanB and variants thereof. In one especially preferred embodiment the transporter domain is a transporter domain of the autodisplay system, also referred to as autotransporter pathway, of the AIDA-I type of gram-negative bacterial cells, in particular of E. coli or a variant thereof, for example those described by Niewert et al. (2001).

Variants of the autotransporter sequences mentioned above can be obtained, for example, by altering the amino acid sequence in the loop structures of the β-barrel that do not belong to the transmembrane sections. Optionally, the nucleic acids coding for the surface loops can be deleted entirely. In addition, conservative amino acid exchanges can be made within the amphipathic β-folded sheet structures, i.e. exchange of a hydrophilic amino acid for another hydrophilic amino acid and/or exchange of a hydrophobic amino acid for another hydrophobic amino acid. Preferably a variant has, at the amino acid level, a sequence identity of at least 70%, at least 80%, at least 90%, at least 95% or at least 98% with the corresponding naturally-occurring sequence of the autotransporter domain, especially in the region of the β-folded sheet structures.

As described above, the problem of the present invention is solved, in one aspect, by a micro-organism which expresses a polypeptide on its surface, as described above, or has been transformed using a nucleic acid molecule, as described above.

In one preferred embodiment, the term “micro-organism”, which is used interchangeably with the term “host cell”, as used here, refers to a micro-organism which is capable of expressing a polypeptide. Such a micro-organism can also be referred to as a “whole-cell catalyst” or “whole-cell biocatalyst”. In another preferred embodiment the cell or host cell is a prokaryotic cell, preferably a gram-negative bacterial cell, quite especially preferably an E. coli cell. In another preferred embodiment the cell is a eukaryotic cell. In another preferred embodiment the cell or host cell is a spore of a prokaryote.

A bacterial cell comprises a number of compartments, which are separated from one another by hydrophobic membranes. A gram-positive bacterial cell has a plasma membrane, which delimits the cytosol, the interior of the cell. The plasma membrane is surrounded by a peptidoglycan layer. In contrast, gram-negative bacteria possess, in addition to the plasma membrane, another membrane known as the outer membrane. The term “surface”, as used here, preferably refers to a layer of the microorganism that is exposed to the environment, wherein the environment is, for example, the liquid culture medium used as culture for the cell of interest. In one quite especially preferred embodiment, a polypeptide according to the present invention is expressed on the outside of the outer membrane of a gram-negative bacterial cell. In one preferred embodiment a polypeptide according to the present invention is expressed on the inside of the outer membrane of a gram-negative bacterial cell. In another preferred embodiment the polypeptide according to the invention is expressed on the outside of a spheroplast, which is a gram-negative bacterial cell from which the outer membrane has been removed. The person skilled in the art is familiar with methods that can be used for producing spheroplasts. In another preferred embodiment, the polypeptide according to the invention is expressed on the outside of a gram-positive bacterial cell. Thus, as the terms “displayed on the surface” and “expressed on the surface” are used here, they are synonymous.

According to a further aspect, the problem of the present invention is solved by a membrane fraction, which can be obtained from the cell according to the previous aspect of the present invention.

In one preferred embodiment of the present invention, the membrane fraction or the membrane preparation, the two terms being used interchangeably, comprises a cellulase according to the first aspect of the invention, preferably in a catalytically active state. The terms “membrane fraction” and “membrane preparation”, as used here, preferably refer to a product that is enriched in membrane constituents, preferably constituents of the outer membrane of a gram-negative bacterium. The person skilled in the art is familiar with protocols and methods that can be used for producing membrane preparations. For example, bacterial cells can be harvested from a culture and submitted to lysis, for example by cycles of freezing and thawing, acoustic irradiation, resuspension in lysis buffer or the like, followed by differential centrifugation, in order to isolate membrane fractions of the cells. In one preferred embodiment of the present invention, the membrane preparation is a preparation of the outer membrane, i.e. a preparation in which there is enrichment of the constituents of the outer membrane, relative to the constituents of other membranes and compartments, such as the cytosol, the inner membrane and the periplasm. The person skilled in the art is familiar with protocols and methods that can be used for isolating or enriching the outer membrane or constituents thereof, for example a lysozyme treatment of bacterial cells, followed by centrifugation steps. In one preferred embodiment, the membrane fraction can be a treated membrane fraction, i.e. the content or the properties of the membrane fraction have been modified, for example by purifying a protein constituent of the membrane fraction or by solubilising the membrane fractions and/or taking up constituents of the membrane fraction in vesicles. In one preferred embodiment of the present invention, the membrane fraction can be immobilised—for example on the surface of a vessel or a column.

According to a further aspect, the problem of the present invention is solved by a method for producing a reaction product using at least one cellulase, comprising the following steps:

(i) preparation of at least one micro-organism which presents a cellulase on its surface, and/or a membrane preparation of this micro-organism, and (ii) bringing the at least one micro-organism and/or membrane preparation into contact with one or more cellulase substrates under conditions compatible with cellulase activity. The expression “presented” is to be understood, in connection with the present invention, to mean that the cellulase projects into the medium surrounding the micro-organism, or the microorganisms.

The method according to the invention can be especially advantageously applied if the product of the reaction catalysed by the cellulase is a mono-, di- or oligosaccharide, and if the at least one cellulase substrate is a polysaccharide source, especially preferably a cellulose source. The terms “polysaccharide source” and “cellulose source” are to be understood to mean that the materials contain polysaccharides or cellulose, preferably as main component, but do not necessarily consist solely thereof. If the cellulose source is cellulose, depending on the cellulase used, the resulting products of glucose, cellobiose and glucose-based oligosaccharides can be obtained.

Furthermore, the method according to the invention can be advantageously further applied if it includes a step (iii) of the recovery of the micro-organism used in step (ii). The recovery can be done, for example, be centrifuging off the cells from the reaction mixture.

Within the scope of the present invention, it has emerged that step (ii) should preferably be carried out in a pH-range of 4.5 to 6.5, especially preferably in a range of 5.5 to 6.5. Cellulases exhibit their highest activity for corresponding pH-values. Furthermore, it has also proven useful to select the temperature during step (ii) in the range of 30 to 80° C., preferably in the range of 50 to 65° C., since cellulases exhibit the highest level of activity in this temperature range.

If, in the methods described above, cellulases which do not produce glucose as end product have been used, it can be useful to convert the products produced into glucose, since in the presence of larger volumes of cellobiose, the activity of enzymes can be impaired. Within the scope of the present invention, the addition of glucosidases in step (ii) of the method described above is therefore preferred. These glucosidases are advantageously not enzymes linked to micro-organisms, especially preferably these are beta-glucosidase from almonds.

Any polysaccharides can be used as polysaccharide source which are the product of cellulase decomposition. Within the scope of the present invention, however, it has turned out to be especially advantageous if a cellulose source is used as polysaccharide source. The cellulose source can be either purified cellulose with only small proportions of foreign substances (preferably in the range of less than 80%/wt, in particular in the range of less than 90%/wt.). In particular, it can be communal waste water which can be used directly or from which the cellulose can be recovered using known methods and then can be converted with the micro-organism(s). Equally, cellulose-containing waste water from wood pulp works or paper mills can be utilised as cellulose source. It is, however, also possible to use a cellulose source which contains contaminants, such as for example wood which may have been pre-treated. The wood may advantageously be wood, selected from pine, larch, spruce, beech, eucalyptus, fir, poplar, pine, beech, oak, ash, chestnut, birch, cherry, maple or walnut. A preferred wood within the scope of the invention is poplar wood. Alternatively, however, remains of annual plants containing cellulose, for example straw, can be used in the method according to the invention as polysaccharide source. The corresponding wood or plant remains can be pre-treated, for example, to remove any hemicellulose they may contain. The polysaccharide source can, however, contain significant quantities of lignin, and it is also possible to use wood or plant remains which contain hemicellulose.

In the method according to the invention, a type of micro-organisms which presents a specific class of cellulases on its surface can be used, but it is also possible to use a mixture of micro-organisms which present various classes of cellulases on their surface. It is preferable if, in the method, at least one mixture of at least one endo- and at least one exocellulase is used, especially preferably a mixture of at least one endo- and at least one exocellulase and at least one beta-glucosidase. In one especially preferred embodiment, the ratio of endo- to exocellulase-modified micro-organisms is 25:75 to 75:25, and most especially preferably 25:75 to 40:60, the mixture being free from beta-glucosidase-modified micro-organisms. In another preferred embodiment, the method uses a mixture of endo- and exocellulase and beta-glucosidase-modified micro-organisms, which especially preferably are present in the ratio of about 1:2:1. The endocellulase preferably includes a peptide sequence SEQ ID No:2. The exocellulase, independently of this, preferably includes a peptide sequence SEQ ID No:4. The beta-glucosidase, independently of this, preferably includes a peptide sequence SEQ ID No:6.

A further aspect of the present invention concerns the use of micro-organisms, as described above, or membrane fractions, as also described above, to produce a product of a reaction which is catalysed by a cellulase. In one especially preferred embodiment, the product is a degradation product of cellulose, in particular glucose and/or cellobiose.

In another preferred embodiment, the problem of the present invention is solved by a method for producing a micro-organism which presents a cellulase on its surface, and comprising the steps:

(a) inserting a nucleic acid molecule as described above containing an expression control sequence operatively linked to the nucleic acid molecule, into a micro-organism, and optionally

(b) bringing the micro-organism into contact with a substance activating the expression control sequence.

Equally, the problem of the present invention is solved by a method for producing a membrane preparation which includes, in addition to the steps (a) and (b) described above, a subsequent step of obtaining the membrane preparation from the micro-organism. The measures necessary for this step are the same as those described previously.

In another preferred embodiment, the problem of the present invention is solved by a composition which contains one or more of the micro-organisms described above which present cellulases on their surface and/or a corresponding membrane preparation.

The present invention is also illustrated by the following figures, the examples, which are not to be regarded as limitative, the further features, embodiments, principal points and advantages of which can be derived from the invention.

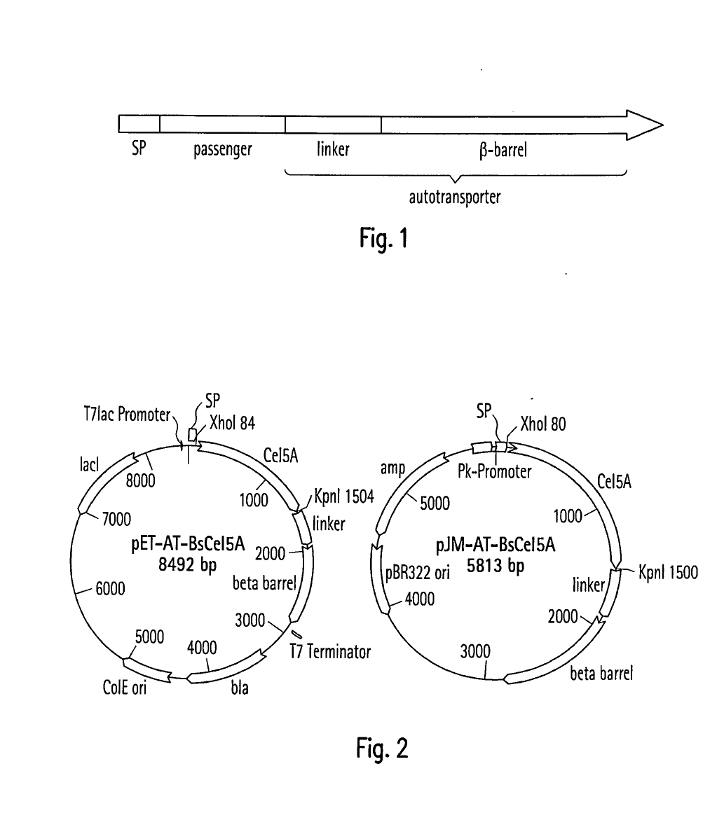

FIG. 1: Schematic structure of the autodisplay cassette. The signal peptide (SP) serves as transport across the inner membrane. The beta-barrel folds itself into the outer membrane and anchors the construct there. The linker is needed to guarantee complete translocation of the passenger, in this case CipA, onto the cell surface.

FIG. 2: Restriction card of the plasmid for the expression of the Cel5A in the inducible T7-system (left) and in the constitutive system (right). The left plasmid is a derivative of pET-11d (Novagen) and has been transformed into the E. coli strain BL21(DE3), the right plasmid is a derivative of pJM007 (Maurer et al., 1997) and has been transformed into E. coli BL21.

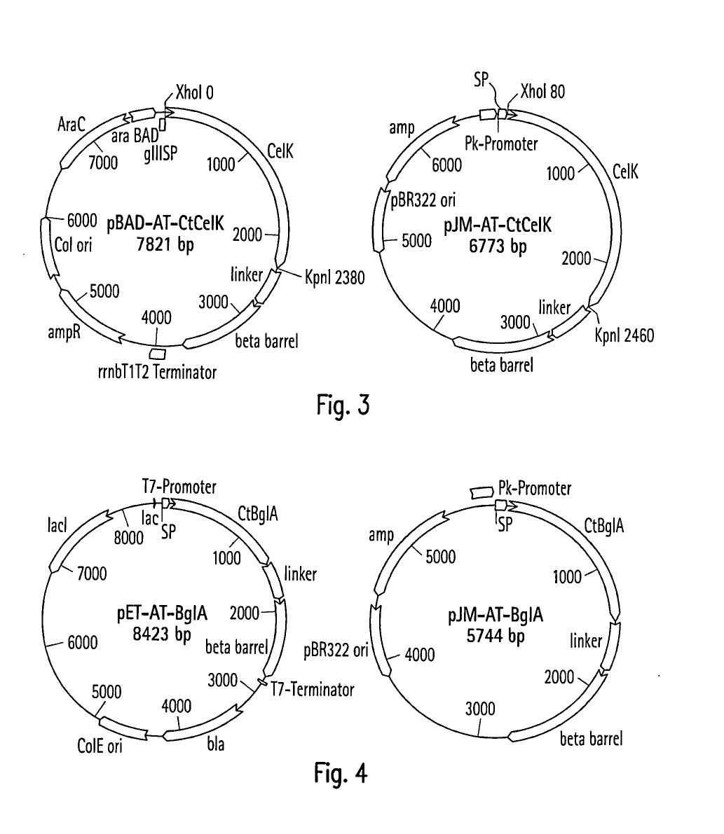

FIG. 3: Restriction card of the plasmid for the expression of the CelK fusion protein in the inducible pBAD-system (left) and in the constitutive system (right). The left plasmid is a pBAD/gIII A derivative (Invitrogen) and has been transformed into E. coli BL21, while the right plasmid is a derivative of pJM007 (Maurer et al., 1997) and has been transformed into E. coli BL21.

FIG. 4: Restriction card of the plasmid for the expression of the BglA fusion protein in the inducible T7-system (left) and in the constitutive system (right). The left plasmid is a derivative of pET-11d (Novagen) and has been transformed into the E. coli strain BL21(DE3), while the right plasmid is a derivative of pJM007 (Maurer et al., 1997) and has been transformed into E. coli BL21.

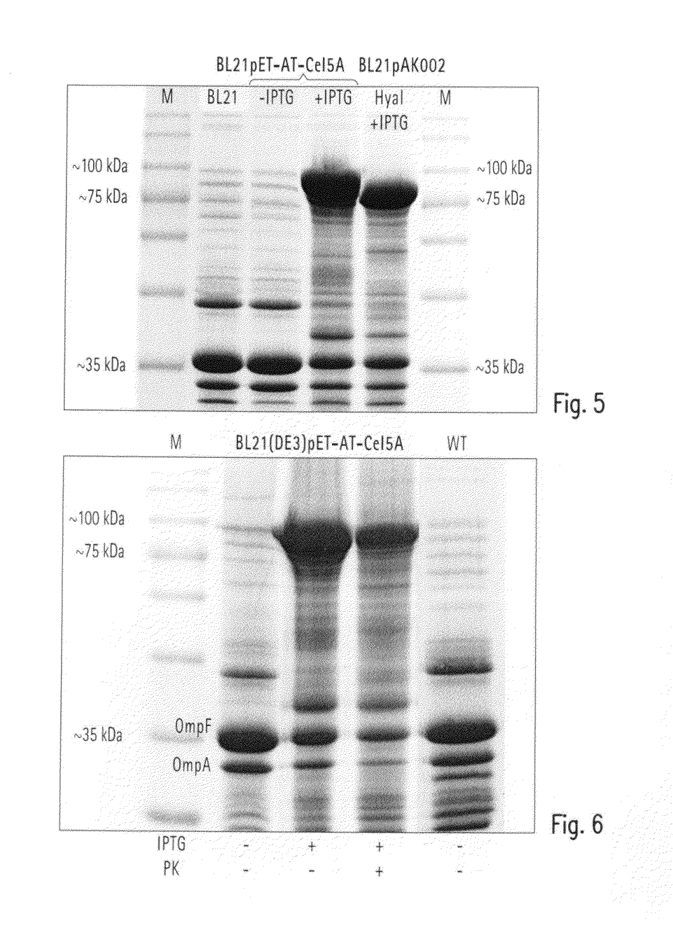

FIG. 5: Comparison of the expression of the Cel5A fusion protein and of the fusion protein of the nonsense control (NC) on the surface of E. coli BL21(DE3). Outer membrane proteins have been isolated by means of differential cell fractionation, separated using SDS-PAGE (10% acrylamide) and stained using Coomassie Brilliant Blue. M: Protein size standard. −/+IPTG: not induced or induced; WT: host strain.

FIG. 6: Proof of the surface display of the Cel5A fusion protein on E. coli BL21(DE3). The outer membrane proteins have been isolated by means of differential cell fractionation, separated using SDS-PAGE (10% acrylamide) and stained using Coomassie Brilliant Blue. M: Protein size standard; WT: host strain, PK: proteinase K. The digestion of whole cells with PK took place before isolation of the outer membrane protein.

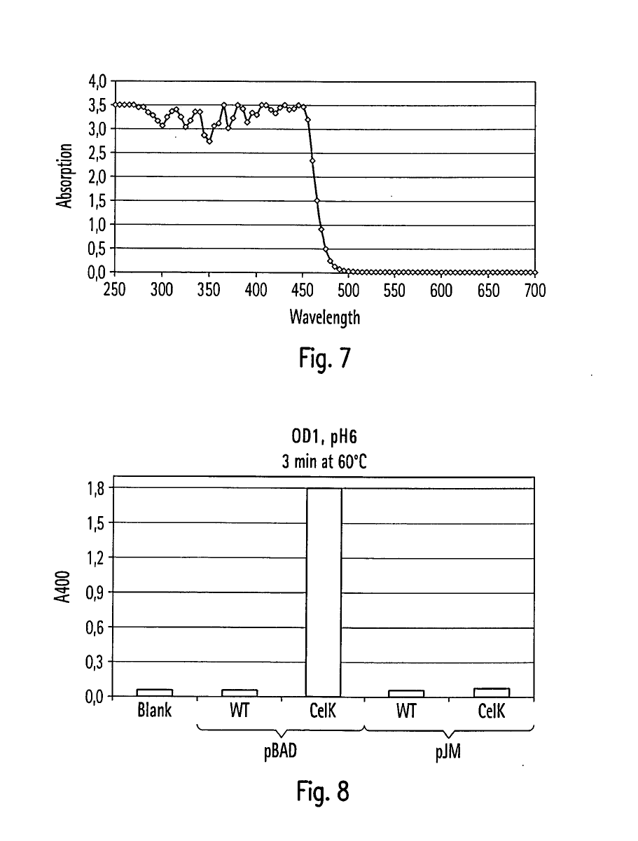

FIG. 7: Spectrum of p-nitrophenolate under CelK conditions. 5 mM p-nitrophenol was photometrically determined in Na-citrate buffer (pH 6) after 10 minutes incubation at 60° C. and subsequent alkalinisation.

FIG. 8: CelK activity inducible (pBAD) versus constitutive (pJM). The cells were incubated with p-nitrophenol-cellobioside for 3 min at 60° C. and pH 6. After incubation, alkalinisation took place using sodium carbonate. The resulting p-nitrophenolate was detected at 400 nm. Blank: buffer+substrate (without cells). WT: host strain (without plasmid).

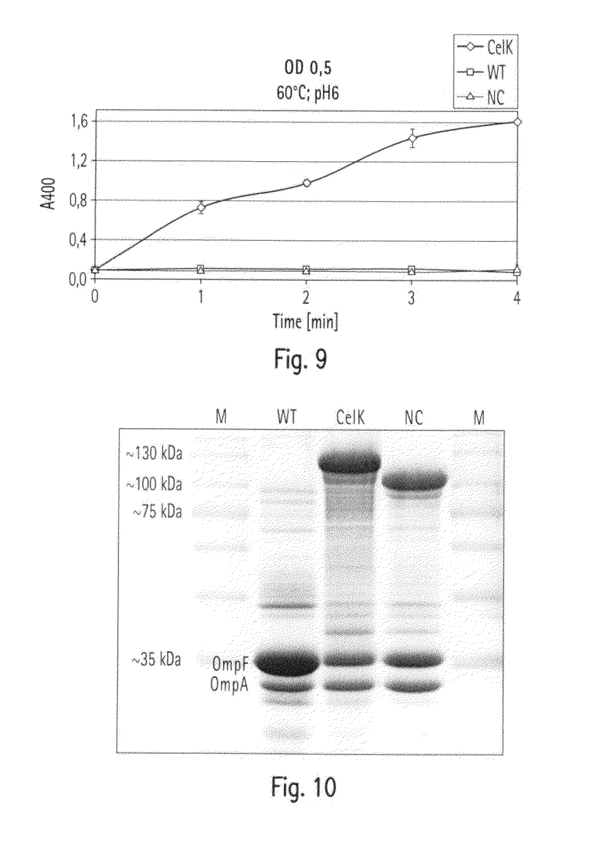

FIG. 9: Nonsense control (NC) for CelK activity. The cells were mixed with p-nitrophenol-cellobioside, and incubated at 60° C. and pH 6. The p-nitrophenolate formed was detected at 400 nm after 1, 2, 3 and 4 min. Alkalinisation took place beforehand using sodium carbonate. WT: host strain. The figure shows MW±SD; n=2.

FIG. 10: Comparison of the expression of the CelK fusion protein and of the fusion protein from the nonsense control (NC) on E. coli BL21. Outer membrane proteins were isolated using differential cell fractionation, separated using SDS-PAGE (10% acrylamide) and stained using Coomassie Brilliant Blue. M: Protein size standard; WT: host strain.

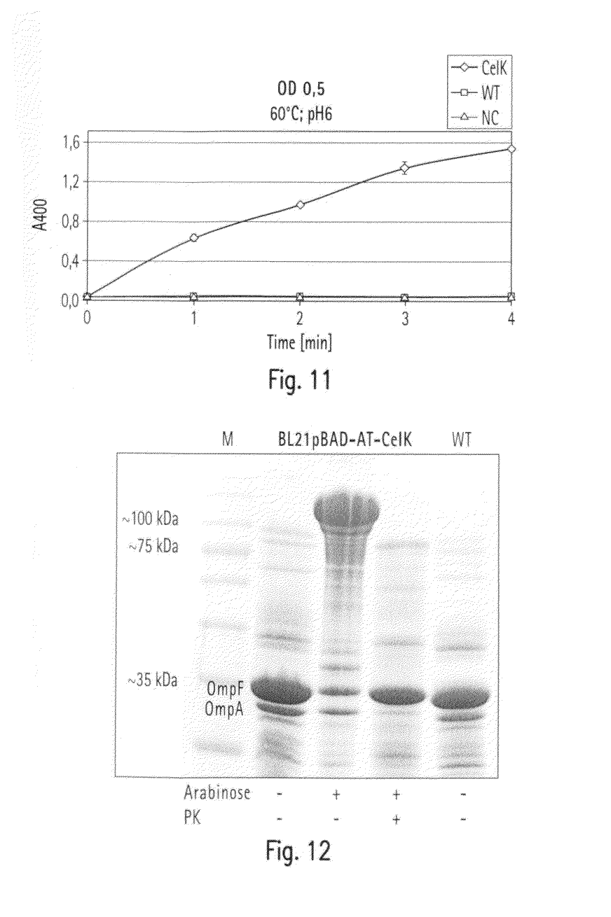

FIG. 11: Storage stability of the CelK strain. The cells were stored for 8 days at 4° C. and the CelK activity was then determined at 60° C. and pH 6 via the increase in the p-nitro-phenolate produced at 400 nm. WT: host strain; NC: Nonsense control. The figure shows MW±SD; n=3.

FIG. 12: Proof of the surface display of the CelK fusion protein on E. coli BL21. The outer membrane proteins were isolated using differential cell fractionation, separated using SDS-PAGE (10% acrylamide) and stained using Coomassie Brilliant Blue. M: Protein size standard; WT: host strain; PK: Proteinase K. The digestion of whole cells with PK took place before the isolation of the outer membrane protein.

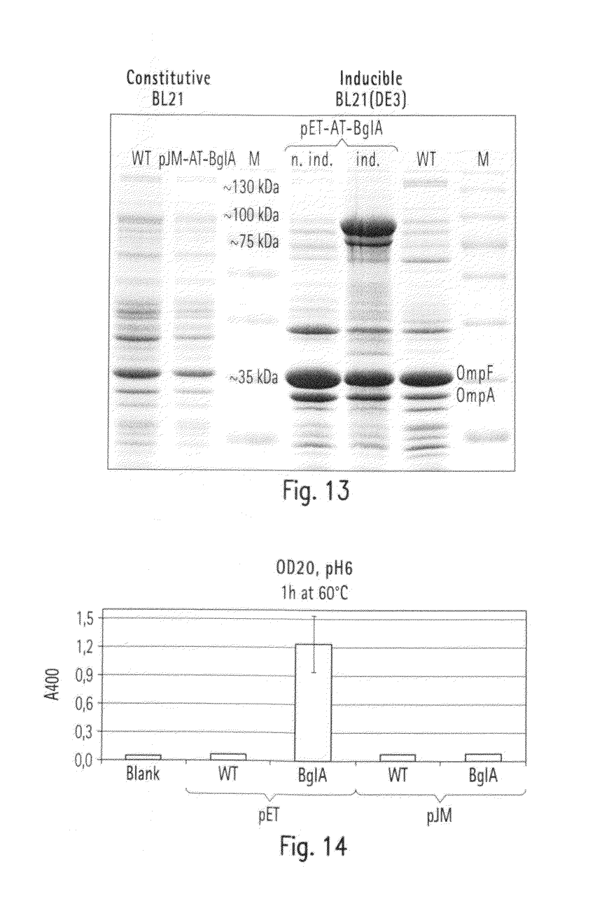

FIG. 13: Expression of the BglA fusion protein in the inducible pET system and constitutive NM system on the surface of E. coli BL21(DE3) and E. coli BL21. Outer membrane proteins were isolated using differential cell fractionation, separated using SDS-PAGE (10% acrylamide) and stained using Coomassie Brilliant Blue. WT: host strain; n. ind and ind.: −/+IPTG; M: Protein size standard.

FIG. 14: BglA activity inducible (pET) versus constitutive (pJM). The cells were incubated using p-nitrophenol-glucopyranoside for 1 h at 60° C. and pH 6. Following incubation, alkalinisation took place with sodium carbonate. The resulting p-nitrophenolate was detected at 400 nm. Blank: buffer+substrate (without cells). WT: host strain (without plasmid). The figure shows MW±SD; n=3.

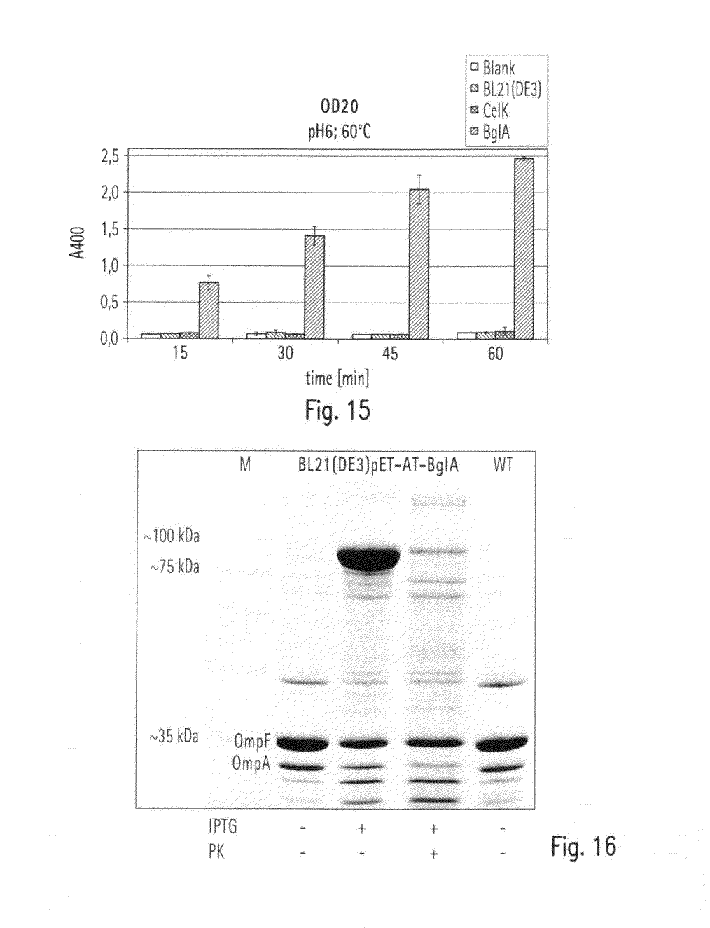

FIG. 15: BglA activity and nonsense control (CelK). The cells were mixed with p-nitrophenol-glucopyranoside and incubated at 60° C. and pH 6. The resulting p-nitrophenolate was detected after 15, 30, 45 and 60 min at 400 nm. Alkalinisation took place beforehand with sodium carbonate. The host strain E. coli BL21(DE3) was used as control. Blank: buffer+substrate (without cells). The figure shows MW±SD; n=3.

FIG. 16: Proof of the surface display of the BglA fusion protein on E. coli BL21(DE3). The outer membrane proteins were isolated using differential cell fractionation, separated using SDS-PAGE (10% acrylamide) and stained using Coomassie Brilliant Blue. M: Protein size standard; WT: host strain; PK: Proteinase K. The digestion of whole cells with PK took place before the outer membrane protein isolation.

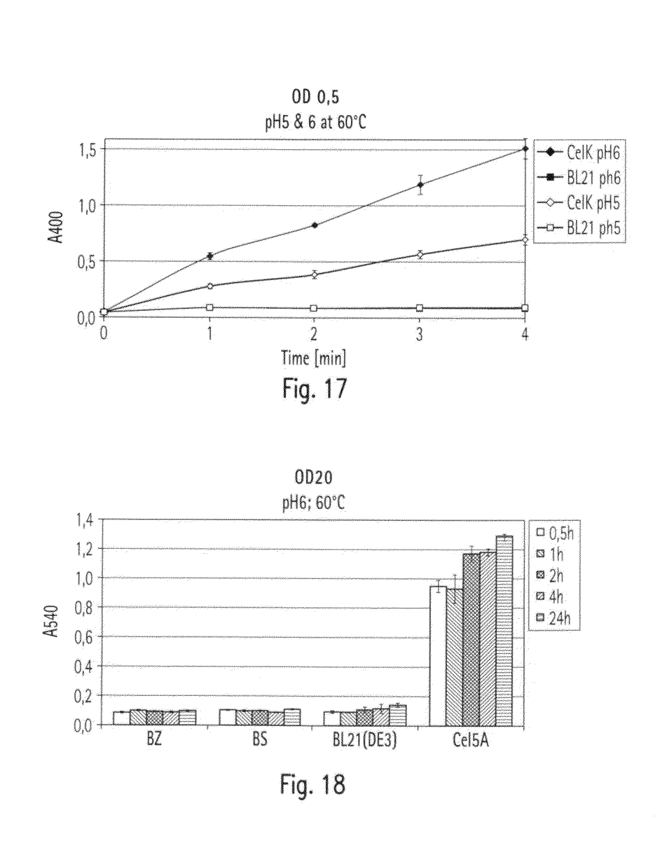

FIG. 17: CelK activity at pH 5 and pH 6. The CelK activity was determined at 60° C. and pH 5 and pH 6 via the increase in the p-nitrophenolate at 400 nm. P-nitrophenol-cellobioside was used as substrate and the host strain E. coli BL21 was used as control. The figure shows MW±SD; n=3.

FIG. 18: CMCase-activity of the Cel5A strain. The Cel5A strain and the host strain E. coli BL21(DE3) were mixed with 1% CMC and incubated at 60° C. and pH 6. After varying incubation periods the reducing sugars formed were detected by means of DNS assay at 540 nm. Blank cells (BZ): cells only (without CMC); Blank substrate (BS): CMC+buffer only (without cells). The figure shows MW±SD; n=3.

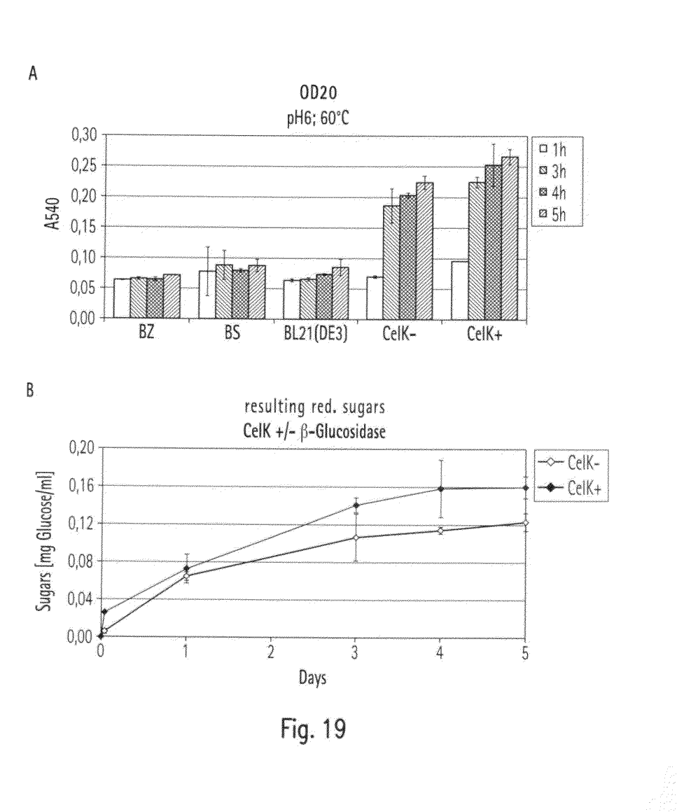

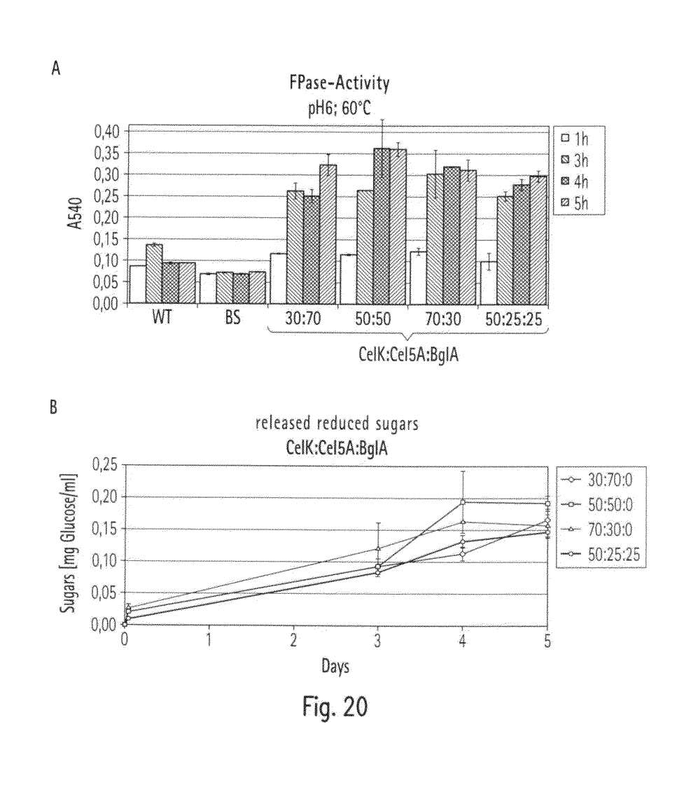

FIG. 19: Avicelase activity of the CelK strain in presence and absence of a beta-glucosidase. The CelK strain was mixed, in the presence and absence of a beta-glucosidase (+/−) made from almonds with 1% Avicel and incubated at 60° C. and pH 6. After varying intervals, the reducing sugars formed were detected by DNS assay at 540 nm. The host strain E. coli BL21 was used as control. A: Absorption values at 540 nm. Blank cells (BZ): cells only (without Avicel); Blank substrate (BS): Avicel+buffer only (without cells). B: Reducing sugars formed by the CelK strain in mg glucose/ml. The figure shows MW±SD; n=3.

FIG. 20: Filter Paper (FP) activity of the autodisplay cellulases at microtitre plate scale. The cellulases were incubated in various ratios with an FP plate (2.4 mg) at 60° C. Mixtures without BglA contained 0.5 U/ml beta-glucosidase from almonds. After varying incubation periods the reducing sugars formed were detected by means of DNS-assay at 540 nm. A: Absorption values at 540 nm. WT: host strain; blank substrate (BS): FP+buffer (without cells). B: Reducing sugars formed by the cellulases in mg glucose/ml. The figure shows MW±SD; n=2.

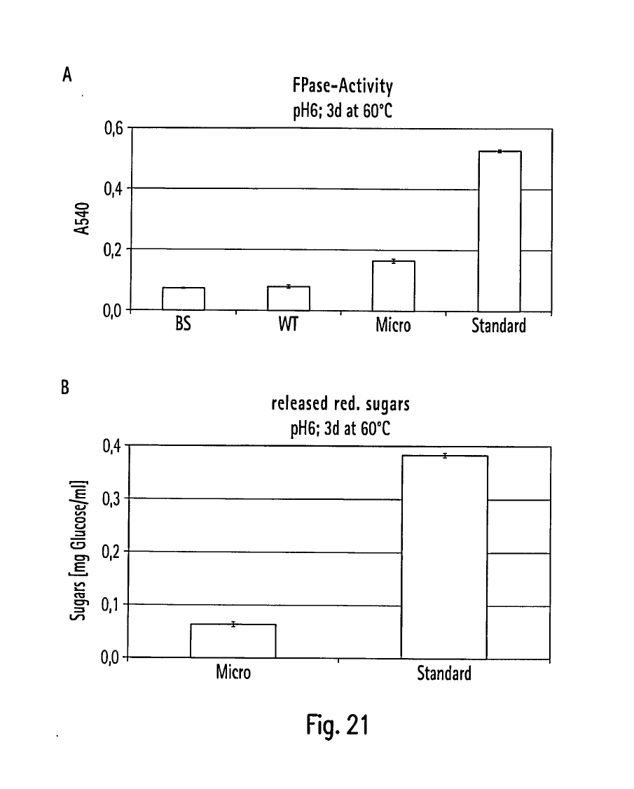

FIG. 21: Filter Paper (FP) activity of the autodisplay cellulases: Microtitre plate scale vs. standard. The cellulases were mixed in equal parts and incubated with either a 2.4 mg filter paper plate (micro) or with a 50 mg filter paper strip (standard) for 3 days at 60° C. The reducing sugars formed were detected by means of DNS assay at 540 nm. A: Absorption values at 540 nm. WT: host strain; blank substrate (BS): FP+buffer (without cells). B: Reducing sugars formed by the cellulases in mg glucose/ml. The figure shows MW±SD; n=2.

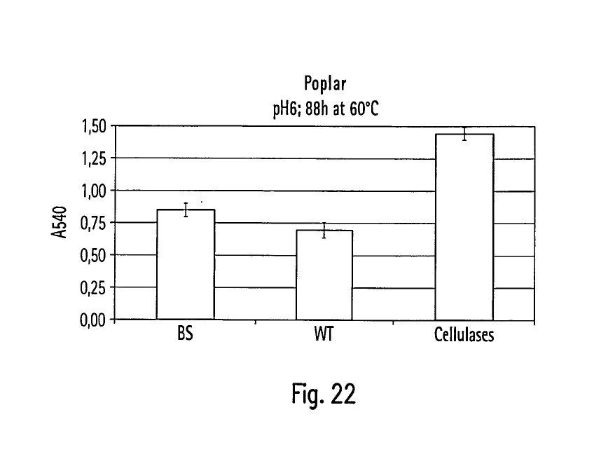

FIG. 22: Poplar degradation by autodisplay cellulases. The cellulases were mixed in equal parts and incubated with 4% poplar for 88 h at 60° C. The reducing sugars formed were detected by means of DNS assay at 540 nm. The figure shows MW±SD; n=2.

### EXAMPLES

### General

The cellulases were ligated as “passengers” in the autodisplay cassette, so that the protein is expressed as fusion protein. FIG. 1 shows the schematic structure of such a construct. The autodisplay cassette is found in various expression systems, so that for the cloning of the cellulase passengers, firstly two inducible autodisplay systems were used—IPTG (pET-derivative) and arabinose (pBAD derivative)—and a constitutive (NM derivative).

The gene sequences of the three cellulases were adapted to the “codon-usage” of E. coli and synthesised by Life Technologies (GeneArt® Gene Synthesis). The optimised gene sequences and the amino acid sequences derived therefrom are in the sequence protocol. The genes were inserted into the autodisplay vectors using the attached Xho I and Kpn I interfaces. For each cellulase, both an inducible and a constitutive autodisplay system was used. In the case of the inducible system, the pET-autodisplay vector was used for the endocellulase Cel5A and the β-glucosidase BglA. Since CelK, at 2.4 kb, is very large, the pBAD autodisplay vector was used for CelK, since this is smaller by comparison with the pET autodisplay vector. The autodisplay cellulase plasmids generated are thus all <10 kb and are summarised in Table 1. The respective plasmid cards are shown in FIGS. 2 to 4.

TABLE 1

| Cellu- | Gene | | Fp | |
|---|---|---|---|---|
| lase | (bp) | Plasmid | (kDa) | E. coli strain |
| Cel5A | 1422 | pET-AT-Cel5A | 102 | E. coli BL21(DE3)pET-AT- |
| | | | | Cel5A |
| | | pJM-AT-Cel5A | | E. coli BL21pJM-AT-Cel5A |
| CelK | 2382 | pBAD-AT-CelK | 139 | E. coli BL21pBAD-AT-CelK |
| | | pJM-AT-CelK | | E. coli BL21pJM-AT-CelK |
| BglA | 1353 | pET-AT-BglA | 101 | E. coli BL21(DE3)pET-AT- |
| | | | | BglA |
| | | pJM-AT-BglA | | E. coli BL21pJM-AT-BglA |

The plasmids inducible by IPTG (pET-derivatives) were transformed into E. coli BL21(DE3) (B, F-, dcm, ompT, Ion, hsdS (rB- mB-) gal, λ (DE3)). For the plasmids inducible by arabinose (pBAD-derivatives) and constitutive (NM-derivatives) E. coli BL21 (B, F-, dcm, ompT, Ion, hsdS (rB- mB-) gal) was used. The E. coli strains generated are also listed in Table 1.

### Expression Conditions for Cellulases

The expression of the cellulase fusion proteins was induced either by adding IPTG or with L-arabinose. In the case of constitutive expression, no inductor needs to be added. The expression conditions of the cellulase strains generated (cf. Table 1) are listed in Table 2. IPTG or L-arabinose was added as soon as the main culture reached an OD₅₇₈ of 0.5-0.6.

TABLE 2

| Strain | Expression of cellulase fusion protein |
|---|---|
| E. coli BL21(DE3)pET- | Induction with 1 mM IPGT for 1 h |
| AT-derivatives | at 30° C. |
| E. coli BL21pBAD-AT- | Induction with 0.2% L-arabinose |
| derivatives | for 4 h at 37° C. |
| E. coli BL21pJM-AT- | 24 h cultivation at 37° C. |
| derivatives | (constitutive expression) |

In order to exclude any influence of periplasmic disulphide bridges on the cellulase transport, the cells were cultivated with 10 mM β-mercaptoethanol (ME). Cells cultivated without ME were used as comparison.

### Example 1

### Endocellulase Cel5A

The Cel5A strains (E. coli BL21(DE3)pET-AT-Cel5A and E. coli BL21pJM-AT-Cel5A) were grown in accordance with Table 2 without and with ME and prepared for either an activity test or for an isolation of the outer membrane proteins.

Cel5A-Activity: Inducible Vs. Constitutive

The activity of the endocellulase was detected using the CMC plate assay. This involves producing agar plates consisting of assay buffer and substrate (carboxymethyl-cellulose). 10 μl cells (OD₅₇₈20) are dripped onto each of the plates, and then incubated at 50° C. The CMC plates are stained using Congo red, which stains polysaccharide chains with n>6 monomers. To finish, the plates are decoloured using NaCl. If CMC breaks down, a light halo forms around the cells, while non-hydrolysed CMC molecules continue to provide a dark-red stain.

In order to determine the optimal incubation period at 50° C., a time series from 0.5-2 h was selected. In addition to the cellulase strains, the host was also tested for CMC degradation. The results showed that the inducible Cel5A strain causes a time-dependent CMC degradation (the haloes become larger as the incubation period increases). Contrary to this, no CMC degradation was detected with the constitutive Cel5A strain. As anticipated, the host showed no CMC degradation. The addition of ME during growth had no effect.

The experiment showed that CMC degradation is possible with the inducible Cel5A-strain. The growth of the cells can occur without adding mercaptoethanol. An incubation period of 30 min for the CMC plates is sufficient.

### Cel5A-Activity: Nonsense Control

In order to exclude any influence of the β-barrel on the Cel5A activity, the CMC plate assay was also conducted with cells which carry a different passenger on the surface which is not capable of degrading CMC. This strain is henceforth referred to as a nonsense control. A test was also made as to whether the promoter is “leaky”. This means that the Cel5A fusion protein would be expressed even without the addition of the inductor. To do so, the Cel5A strain was not induced and the activity was compared with induced cells. The test showed that the non-induced Cel5A strain exhibits slight CMC degradation. This shows that without inductor, a certain amount of expression of the Cel5A fusion protein occurs. The CMC degradation of the induced Cel5A strain is, however, more strongly marked. The nonsense control shows no CMC degradation, exactly as does the host strain without plasmid. The very slightly marked halo in the nonsense control and host is comparable with the buffer sample. So this is a non-specific reaction of the buffer to the agar plate.

In order to guarantee that the number of β-barrel molecules of the Cel5A strain and the nonsense control is comparable, the outer membrane proteins were isolated from the tested cells and separated using SDS-PAGE. The E. coli outer membrane proteins OmpF (37 kDa) and OmpA (35 kDa) are used as marker proteins for the outer membrane fraction. The result is shown in FIG. 5. After induction with IPTG, comparably strong bands for the Cel5A fusion protein and the fusion protein of the nonsense control were detected which correspond well to the calculated MW of approx. 102 kDa and 96 kDa. With the non-induced Cel5A strain, no bands were detected for the Cel5A fusion protein. Equally, as anticipated, the host showed no bands for the Cel5A-fusion protein and the nonsense control.

It was therefore shown that CMC degradation caused by the Cel5A strain involves a specific cellulase reaction.

### Storage Stability of the Cel5A Strain

In order to test whether the Cel5A strain can be stored at 4° C., the activity of the cells from test 3.2 was determined after 10 days in storage. It was apparent from the test that the stored Cel5A strain exhibits no loss of activity in this period. The CMC degradation is comparable with fresh cells. The host and the nonsense control showed no CMC degradation.

The test showed that it is possible to store the Cel5A strain for at least 10 days at 4° C. with no loss of activity.

### Surface Display of the Cel5A Strain

The successful expression of the Cel5A fusion protein has been shown above. The detection of the fusion protein in the outer membrane, however, does not yet offer any conclusion as to its orientation. The enzymes can, as desired, be oriented towards the extracellular space or towards the periplasm. By incubating the intact bacteria cells with proteinase K followed by preparation of the bacterial outer membrane proteins, it is possible to detect the surface display of an autodisplay passenger. Since proteinase K molecules are incapable, because of their size, of passing through the outer membrane of E. coli, the incubation of whole cells with these proteases merely results in the digestion of proteins located on the surface of the outer membrane. The outer membrane proteins OmpF and OmpA are protected from protease digestion since they are integral membrane proteins. Their detection after protease treatment can therefore be regarded as an index for the integrity of the cells.

In order to detect surface display, the Cel5A strain was incubated with proteinase K, following induction of the Cel5A expression. Induced cells were used as comparison which had not been treated with the protease. The E. coli host strain and the non-induced Cel5A strain were used as controls. In FIG. 6, the strong expression, already shown, of the Cel5A fusion protein after IPTG induction at approx. 102 kDa can be seen (cf. FIG. 4). As anticipated, neither in the E. coli host strain nor in the non-induced Cel5A strain was expression at approx. 102 kDa detected. Due to the strong Cel5A expression, a complete proteolytic degradation of the Cel5A fusion protein is difficult. The reduction in the Cel5A bands with the simultaneous maintenance of the OmpF- and OmpA-bands following protease treatment speaks clearly in favour of an outward orientation of the Cel5A.

### Example 2

### Exocellulase CelK

The CelK strains (E. coli BL21pBAD-AT-CelK and E. coli BL21pJM-AT-CeI5K) were cultivated in accordance with Table 2 without and with mercaptoethanol and prepared for either an activity test or for isolation of the outer membrane proteins.

CelK Activity: p-Nitrophenolate Assay

The activity of the exocellulase was determined photometrically via the increase in p-nitrophenolate. P-nitrophenol cellobioside was used as substrate. In the literature an absorption maximum of 380-420 nm is described for the p-nitrophenolate. In order to determine the optimal wavelength for the measurement of p-nitrophenolate, firstly a spectrum was recorded under CelK assay conditions. To do so, p-nitrophenol was added to sodium citrate buffer pH 6 and incubated at 60° C. Since the yellow colouring of the p-nitrophenolate is most strongly marked when alkaline, the sample was further alkalinised with sodium carbonate and then measured photometrically in the microtitre plate. FIG. 7 shows that under CelK conditions a p-nitrophenolate measurement is possible at approx. 360-450 nm. Based on the literature, an absorption measurement at 400 nm was defined for the following CelK assay.

In order to determine the activity of the CelK strain, a standard curve was made with p-nitro-phenolate (0 mM-1 mM). The p-nitrophenolate samples were treated in the same way as the CelK samples and measured undiluted and diluted 1:2 respectively.

### CelK Activity: Constitutive Versus Inducible

Preliminary CelK tests had shown that the inducible CelK strain exhibits greater activity compared with the constitutive CelK strain. In order to determine a precise ratio, the activities were determined in a direct comparison. The result is shown in FIG. 8. For the inducible CelK strain, using a standard curve, activity of 210 mU/OD₅₇₈1/ml was determined. For the constitutive CelK strain, after 3 min at 60° C., no conversion was observed in the OD₅₇₈1 used. Therefore the constitutive CelK strain (OD₅₇₈1) was incubated for 30 min at 60° C. Now an absorption at 400 nm of 0.25 was determined, which corresponds to an activity of 2.2 mU/OD₅₇₈1/ml. The inducible CelK strain is therefore approx. 100 times more active by comparison with the constitutive CelK strain. The other tests were therefore continued with the inducible CelK strain (cultivation without mercaptoethanol).

### CelK-Activity: Nonsense Control

In the same way as the endocellulase, a nonsense control for activity was also to be tested for the exocellulase. FIG. 12 shows that the OD₅₇₈ of the CelK strain in the mixture should be 1. In the following test, OD₅₇₈0.5 was tested. Since the p-nitrophenolate formation was already very marked after approx. 3 min (cf. FIG. 8), a time series of 0-4 min was selected for this test. To do so, the CelK strain, the nonsense control and the host strain were mixed with p-nitrophenol cellobioside and after each minute the p-nitrophenolate formed was measured at 400 nm. The samples were diluted 1:2 before the alkalinisation. The result is shown in FIG. 9. It emerged that OD₅₇₈0.5 is sufficient for the activity measurement. For the CelK strain, using the standard curve (1:2 diluted) an activity of approx. 300 mU/OD₅₇₈1/ml was calculated. The first 3 min were observed. After that, the reaction is no longer linear. The nonsense control showed, as did the host strain, no conversion of the substrate.

In order to verify whether the expression of the CelK fusion protein and of the fusion protein of the nonsense control is the same, in addition to the activity test, the proteins of the outer membrane were isolated and separated using SDS-PAGE. The E. coli outer membrane proteins OmpF (37 kDa) and OmpA (35 kDa) were used as marker proteins for the outer membrane fraction. The result is shown in FIG. 10. After induction with arabinose, comparably strong bands for the CelK fusion protein and the fusion protein of the nonsense control were detected, which correspond well to the calculated MW of approx. 139 kDa and 103 kDa. No bands of the two fusion proteins were observed in the host strain.

This test showed that the conversion of the p-nitrophenolate is a specific CelK reaction.

### Storage Stability of the CelK Strain

In order to test whether the CelK strain can be stored at 4° C., the activity of the cells from the above test was determined following 8 days of storage. FIG. 11 shows that the stored CelK strain exhibits no loss of activity in this period. The formation of p-nitrophenolate is comparable with fresh cells (FIG. 9). Here, too, activity of approx. 300 mU/OD₅₇₈1/ml was achieved. The host and the nonsense control showed no conversion of the substrate.

The test showed that it is possible to store the CelK strain for at least 8 days at 4° C. with no loss of activity.

### Surface Display of the CelK Strain

The successful expression of the CelK fusion protein has already been shown in FIG. 10. In order to confirm the outward orientation of the enzyme, the CelK strain was treated with proteinase K and then the outer membrane proteins were isolated. OmpF and OmpA were brought in as proof of the integrity of the cells after protease treatment.

The CelK strain was incubated with proteinase K following induction of the CelK expression. Induced cells which had not been treated with protease were used as comparison. The E. coli host strain and the non-induced CelK strain were used as controls. In FIG. 12, the strong expression of the CelK fusion protein already shown, following arabinose induction at approx. 139 kDa, can be seen. As anticipated, neither in the E. coli host strain nor in the non-induced CelK strain was expression at approx. 139 kDa detected. Because of the strong CelK expression a complete proteolytic degradation of the CelK fusion protein is difficult. The CelK band may have completely disappeared, but the OmpA band is also somewhat weaker in comparison with the host strain. Nevertheless this speaks clearly in favour of an outward orientation of the enzyme.

### Example 3

### Beta-Glucosidase BglA

BglA Expression and Activity: Inducible Vs. Constitutive

The expression of the fusion protein, in the case of the constitutive system, took place for 24 h at 37° C. The induction of the expression in the pET system was achieved by means of one-hour incubation of the cells with 1 mM IPTG at 30° C., once they had achieved an OD₅₇₈ of 0.6. There is no need to add 10 mM 6-mercaptoethanol during cultivation, as the BglA amino acid sequence contains only one cysteine and so no disulphide bridges can be formed in the periplasm which could affect the transport.

Once the two BglA strains (induced and constitutive) had been generated, the expression of the BglA fusion protein in the outer membrane of E. coli was to be verified. To do so, the outer membrane proteins of both strains were isolated and separated using SDS-PAGE. The E. coli outer membrane proteins OmpF (37 kDa) and OmpA (35 kDa) were used as marker proteins for the outer membrane fraction. The result is shown in FIG. 13. In the case of the inducible expression system, following induction with IPTG a strong band for the BglA fusion protein was detected, which corresponds well to the calculated MW of approx. 101 kDa. For the non-induced BglA strain and for the host strain, as anticipated, no band could be detected for the BglA fusion protein. In the constitutive system, no expression of the BglA fusion protein was detected. Experience has shown that the detection of a constitutively expressed passenger by means of Coomassie staining is not sensitive enough.

Once the expression of the BglA-fusion protein had been verified, the BglA activity of the constitutive and inducible strain was determined by means of p-nitrophenol-glucopyranoside as substrate. The activity of the BglA strain was determined photometrically via the formation of p-nitro-phenolate at 400 nm. Contrary to the exocellulase CelK, p-nitrophenol-glucopyranoside was used as substrate. In order to establish the assay, the test was first conducted using a bought beta-glucosidase (from almonds). To do so, the optimal conditions were selected for this enzyme (pH 5 and 35° C.). Two different concentrations were used. A concentration-dependent activity of the beta-glucosidase was detected. The blank, as anticipated, shows no increase in p-nitrophenolate.

BglA-activity is determined via the absorption measurement of the p-nitrophenolate produced at 400 nm (yellow discolouration). The reaction took place, on the basis of the published, optimal conditions for the BglA, at a pH value of 6.0 and an incubation temperature of 60° C. Since the incubation period of the BglA strain with the substrate was not known, the reaction mixtures were incubated until a clear yellow colouration (pNitrophenolate) could be seen. In the case of the inducible BglA strain, this was registered after 1 h at 60° C. Before the photometric measurement, the cells were separated and the supernatant transferred into a 96-well microtitre plate. After alkalinisation by adding sodium carbonate, the absorption measurement took place at 400 nm. In FIG. 14 it can be seen that the inducible BglA-strain was capable of converting the pNitrophenol-glucopyranoside (pNitrophenolate increase at 400 nm). Contrary to this, the constitutive BglA-strain showed no formation of p-Nitrophenolate within 1 h at 60° C. A longer incubation period was not tried. Both the blank and the host strain showed no conversion of the substrate. As with the other two cellulases, better activity was achieved with the inducible expression system.

### BglA Activity: Nonsense Control

Once it was shown that the inducible BglA-strain is active, a time series was recorded in order to be able to determine the activity better. At the same time, corresponding to the other two cellulases, a “nonsense control” was also tested. This was an E. coli-strain, which presents a different passenger on the surface, which cannot convert the substrate. The CelK strain was selected. Although this is a cellulase, CelK cannot convert pNitrophenol glucopyranoside. In order to determine the BglA-activity, firstly a pNitrophenol standard curve was recorded. Next, the BglA-strain, the nonsense control (CelK strain) and the host strain were mixed with p-Nitrophenol glucopyranoside and after varying incubation periods at 60° C., the p-Nitrophenolate formed was measured at 400 nm. In FIG. 15, it can be seen that the BglA-strain shows a continuous increase in pNitrophenolate over time. Using the standard curve, a BglA activity of approx. 1 mU/ml/OD1 was calculated. The nonsense control, like the host strain E. coli BL21(DE3), showed no conversion of the substrate.

Surface Display of the BglA on E. coli BL21(DE3)

The successful BglA activity of the inducible strain was shown in FIG. 14, while the expression of the BglA fusion proteins is illustrated in FIG. 13. In order to detect the surface display, the BglA strain was incubated with proteinase K following induction of the BglA expression. The protease, because of its size, is unable to get into the E. coli cell and therefore only degrades proteins which are on the surface of the cell. As comparison, induced cells were used which had not been treated with the protease. The E. coli host strain and the non-induced BglA strain were used as controls. OmpA and OmpF are integral membrane proteins which are protected against protease digestion. These therefore serve as an index for the integrity of the cells. In FIG. 13, the strong expression already shown of the BglA fusion protein following IPTG induction at approx. 101 kDa can be seen. As anticipated, expression at approx. 101 kDa was detected neither in the E. coli host strain nor in the non-induced BglA strain (FIG. 16). Because of the strong BglA expression, complete proteolytic degradation of the BglA fusion protein is difficult. The reduction in BglA bands while the OmpF- and OmpA-bands are maintained following protease treatment speaks, according to our experience, clearly in favour of an outward orientation of the BglA.

### Example 4

With the aid of the autodisplay technology, it was possible to present three active cellulases on the surface of E. coli. In all cases, an inducible expression system was the best. The cellulase strains and their cultivation are summarised in Table 2.

In order to use the autodisplay cellulases together for a synergistic cellulose degradation, common parameters such as pH-value and temperature must be defined. Exocellulase and β-glucosidase have a pH optimum of 6.0. The optimum temperature is 60° C. However, endocellulase has a pH optimum of 5.0 and an optimum temperature of 50° C.

### Determination of the Common Temperature

Since two of the three cellulases (CelK and BglA) have an optimum temperature of 60° C., it was necessary to test whether the endocellulase Cel5A is also active at 60° C. In the literature, a slight reduction in Cel5A activity is described (approx. 5-10%) at 60° C., compared with the activity at 50° C. (Lin L et al, 2009). The activity of the Cel5A was determined using the CMC plate tests at pH 5. This was done by dripping Cel5A-cells onto a CMC plate, then incubating for 30 min at 50° C., 55° C. and 60° C. The host strain E. coli BL21(DE3) and buffer were used as control. The CMC plates were stained using Congo red. Finally, the plates were decoloured with NaCl. It was found that the Cel5A cells degrade equally well at every CMC temperature used (light halo), while the host strain and the buffer are negative. The Cel5A cells can thus also be used at 60° C. In the following passage therefore, work with all three cellulases was carried out at 60° C.

### Determination of the Common pH Value

Exocellulase (CelK) and β-glucosidase (BglA) have an optimum pH of 6. However, endocellulase (Cel5A) has an optimum pH of 5. The literature describes a reduction in Cel5A activity at pH 6 compared with pH 5 (Lin L et al, 2009). It was therefore necessary to test whether it is possible to use CelK at pH 5. To do so, the CelK strain, following induction of the protein expression, was firstly harvested in sodium citrate puffer pH 6 and secondly in sodium acetate buffer pH 5. The CelK-activity was determined from the increase in p-nitrophenolate at 400 nm by degradation of the pNitrophenol cellobioside (pNPC) at 60° C. The host strain E. coli BL21 was used as control. FIG. 17 shows that under both pH conditions an increase in pNitrophenolate can be seen, while the host strain remains unaltered. It can also be seen that at pH 6 compared with pH 5, more pNitrophenolate is produced. At pH 6, CelK-activity of on average 270 mU/ml/OD1 is achieved. At pH 5, however, it was only approx. 110 mU/ml/OD1. So a drop in pH in the assay from pH 6 to pH 5 led to a 60% drop in CelK activity.

A loss of activity is also described for the endocellulase Cel5A, when the pH is raised from 5 to 6. But since the β-glucosidase BglA also has an optimum pH of 6, the subsequent assays are conducted at pH 6, so that only one enzyme has to be used under non-optimal pH conditions.

### Detection of Reducing Sugars: Establishment of the DNS Assay

The common cellulase activity of the autodisplay cellulases was next tested on cellulose substrates. This was done i.a. using colorimetry by measuring the reducing sugars released according to the dinitrosalicylic acid method (Miller GL 1959). The method links the oxidation of a carbonyl compound with the reduction of a nitro group in 3,5-dinitrosalicylic acid under alkaline conditions. This involves reducing 3,5-dinitrosalicylic acid to 3-amino-5-nitrosalicylic acid, which has an absorption maximum at 540 nm. In this reaction the colour of the DNS solution changes from orange-yellow to red.

### CMCase-Activity of the Endocellulase Cel5A

So far the Cel5A-activity has been measured indirectly using the CMC plate test. By using the DNS method, the reducing ends of the carboxymethylcellulose formed can be determined quantitatively by the Cel5A reaction. To do so, the Cel5A-strain was mixed with 1% CMC and incubated at 60° C. After varying incubation periods the cells were separated and the supernatant mixed with DNS reagent. Following incubation at 95° C. and 4° C. the absorption at 540 nm was determined using photometry. The host strain E. coli BL21(DE3) was used as control. As controls, only cells (BZ) or only CMC (BS) were used. In FIG. 18 it can be seen that the Cel5A-strain forms reducing ends which can be detected using DNS reagent. Because of the carboxymethyl substitutions, the CMC degradation is not a linear reaction, as the endocellulase is affected by the degree of substitution. Therefore a conversion rate of approx. 5% should not be exceeded. After 30 min at 60° C. the Cel5A strain converts approx. 8% of the CMC. In order to achieve a better estimation of the Cel5A activity, the incubation at 60° C. should be <30 min. The host strain and the other two controls used, as anticipated, show no endocellulase activity.

### Example 5

### Avicelase Activity of the Exocellulase CelK

Exocellulase activity has so far been determined with pNitrophenol-cellobioside. This is a soluble and thus easily convertible substrate. In order to test CelK-activity on crystalline cellulose regions, Avicel (microcrystalline cellulose) was used as substrate. This was done by incubating the CelK strain with 1% Avicel at 60° C. and after varying incubation periods, the reducing sugars released were determined by means of DNS assay at 540 nm. The concentration of the reducing sugars is determined using a glucose standard curve in mg glucose/ml. Since the exocellulase releases cellobiose, a β-glucosidase was added during the Avicel conversion, which degrades the cellobiose into two reducing sugar units in the form of glucose. This enables a more precise determination of the quantity of reducing sugars formed, since this is calculated on the basis of a glucose standard curve. At the same time the cellobiose acts as an inhibitor of the exocellulase, which is removed using the β-glucosidase. The conversion of the Avicel was analysed in the presence and absence of a commercially-available, purified β-glucosidase from almonds. The host strain E. coli BL21 was used as comparison. As controls, only cells (BZ) or only substrate (BS) were used. It can be seen from FIG. 19A that the CelK strain is capable of degrading microcrystalline cellulose. The host strain, as well as the controls used, show no increase in reducing sugars. The concentration of reducing sugars released is shown in FIG. 19B. As expected, there is a higher concentration in the presence of β-glucosidase from almonds, by comparison with the conversion without β-glucosidase.

β-Glucosidase BglA: pNPGase-Activity

The commercially-available cellulase mix Celluclast® (Novozymes) contains cellulases from the fungus Trichoderma reseei ATCC 26921. This mix catalyses the degradation of cellulose into glucose, cellobiose and higher glucose polymers. For the best possible cellulose conversion, the additional use of a β-glucosidase from Aspergillus niger (Novozym© 188) is recommended. The dose described is Novozyme 1% Celluclast and 0.2% Novozym 188. The optimal conditions for Celluclast are a temperature of 50-60° C. and a pH value of 4.5-6.0. Therefore, for comparison, the reaction conditions of the autodisplay cellulases of 60° C. and pH 6 were adopted.

The β-glucosidase activity was determined photometrically (pNPGase-activity) with pNitrophenol-glucopyranoside (pNPG) via the increase in pNitrophenol-glucopyranoside at 400 nm. This was done by incubating the BglA strain (OD₅₇₈20) with pNPG for 15 min at 60° C. The host strain E. coli BL21(DE3) and buffer (blank) were used as control. For comparison 1% Celluclast and 0.2% Novozym188 were incubated with pNPG at 60° C. Since after just 3 min a strong yellow discolouration was seen, the pNitrophenolate content was measured after 3 min, instead of after 15 min, corresponding to the BglA. 1% Celluclast and 0.2% Novo188, by comparison with the BglA strain, which was used in a concentration of OD₅₇₈20, show a stronger increase in pNitrophenolate. The controls, as expected, showed no pNPGase activity.

With the aid of the pNitrophenolate standard, β-glucosidase activity was calculated in U/I (cf. Table 3). Novozym 188 showed a 14-fold better activity by comparison with the BglA strain. Celluclast showed a 12-fold stronger activity.

TABLE 3

| Cellulase | U/I |
|---|---|
| BglA | 22 |
| Novozym 188 | 312 |
| Celluclast | 264 |

Exocellulase CelK: pNPCase Activity

The activity of the exocellulase was determined photometrically, for practical reasons, with pNitrophenol-cellobioside (pNPC) via the increase in pNitrophenolate at 400 nm (pNPCase activity). This was done by incubating the CelK strain (OD₅₇₈0.5) and Celluclast (1%) with pNPC for 4 min at 60° C. The host strain E. coli BL21 and buffer (blank) were used as controls. The CelK strain with an OD₅₇₈0.5 showed a stronger increase in pNitrophenolate compared with 1% Celluclast. Both controls used showed, as expected, no pNPCase activity. With the aid of the pNitrophenolate standard, the CelK-activity was calculated in U/I (cf. Table 4). The CelK strain showed double the pNPCase activity by comparison with Celluclast.

TABLE 4

| Cellulase | U/I |
|---|---|
| CelK | 108 |
| Celluclast | 52 |

### Endocellulase Cel5A: CMCase Activity

Endocellulase activity was calculated with CMC by determining the reducing sugars by means of DNS assay (CMCase activity). This was done by incubating the Cel5A-strain (OD₅₇₈20) and Celluclast 1% with CMC for 15 min at 60° C. The host strain E. coli BL21(DE3) and buffer (blank) were used as controls. With Celluclast, a stronger release of reducing sugars was observed by comparison with the Cel5A-strain. With the aid of the glucose standard curve, the CMCase activity was calculated in U/I (cf. Table 5). Celluclast shows a 2.4 times better CMCase activity compared with the Cel5A-strain. CMC is, however, not specific for endocellulases. Exocellulases and β-glucosidases also release small quantities of reducing sugars from CMC. Since Celluclast is a mix of Trichoderma cellulases, CMC per se is better converted by comparison with the Cel5A-strain, which presents only one endocellulase on the surface. The 2.4 times better CMCase activity of the Celluclast therefore does not equate to a 2.4 times better endocellulase activity.

TABLE 5

| Cellulase | U/I |
|---|---|
| Cel5A | 227 |
| Celluclast | 535 |

### Example 6

### Recycling the Autodisplay Cellulases

One advantage of the autodisplay cellulases compared with soluble enzymes is that it is simple to recover the whole-cell catalyst from the reaction mixture. The cells are sedimented, resuspended in the appropriate buffer system and are then available for further conversion. In order to test whether the recycling process has any effect on the activity of the cellulases, after determining the basic activity (cycle 0) the cellulase strains were recovered a total of two times and mixed with fresh substrate (cycle 1 and 2). The percentage activity of the recycled cellulase strains was determined by comparison with the respective basic activity.

Recycling the β-Glucosidase BglA: pNPGase Activity

β-Glucosidase activity was determined photometrically with pNitrophenol-glucopyranoside (pNPG) via the increase in pNitrophenolate at 400 nm (pNPGase-activity). This was done by incubating the BglA-strain (OD57820) with pNPG for 15 min at pH 6 and 60° C. Next, the cells were sedimented and the activity was measured again following resuspension (cycle 1 and 2). The activity was determined via the increase in p-Nitrophenolate at 400 nm, and represents an average value from three individual measurements. The result of the recycling tests show that the enzyme activity moves, in the scope of the standard deviation, at the same level for all cycles. The recycling therefore had no effect on BlgA activity. The BglA strain can thus be reused at least twice after being recovered from the reaction mixture.

Recycling the Exocellulase CelK: pNPCase Activity

The activity of the exocellulase was determined photometrically with pNitrophenol-cellobioside (pNPC) via the increase in pNitrophenolate at 400 nm (pNPCase-activity). This was done by incubating the CelK strain (OD₅₇₈0.5) with pNPC for 4 min at pH 6 and 60° C. Next, the cells were sedimented and the activity was measured again following resuspension (cycle 1 and 2). The activity was determined via the increase in p-Nitrophenolate at 400 nm, and represents an average value from three individual measurements. The result of the recycling tests show that the enzyme activity moves, in the scope of the standard deviation, at the same level for all cycles. The recycling therefore had no effect on CelK activity. As with the BglA strain, the CelK strain can thus be reused at least twice.

### Recycling the Endocellulase Cel5A: CMCase Activity

Endocellulase activity was calculated by determining the reducing sugars by means of DNS assay (CMCase activity). This was done by incubating the Cel5A-strain (OD₅₇₈20) with CMC for 15 min at pH 6 and 60° C. Next, the cells were sedimented and the activity was measured again following resuspension (cycle 1 and 2). The reducing sugars released were detected by means of DNS assay at 540 nm. The values determined are average values from three individual measurements. The Cel5A activity shows a decrease after the first recovery of approx. 14% (cycle 1). After another recycling (cycle 2) a drop in activity of approx. 63% was recorded, by comparison with the basic activity (cycle 0). Contrary to the BglA- and CelK-strain, the recycling of the Cel5A strain is associated with a reduction in activity. Because of the drop in activity after the first cycle is moderate, however, the Cel5A strain can be re-used at least once.

### Example 7

### Filter Paper Assay (FPA)

The FPA is the standard method for the analysis of cellulase activity as a whole (exo-, endocellulase and β-glucosidase) and was developed by Mandels et al in 1976. According to IUPAC (International Union of Pure and Applied Chemistry) the FPA is conducted using a 1×6 cm filter paper strip (Whatman No. 1) as standard substrate (approx. 50 mg) and 1.5 ml total volume. The international unit for filter paper activity (FPU) is defined as micromol glucose equivalent (reducing sugars) released per minute. With the FPA, not more than 2 mg glucose should be released from 50 mg filter paper (4% conversion) in 60 min. Because of the structure of the cellulose, the degradation is not linear. Therefore only a conversion of up to 4% is described as linear. The reducing sugars formed are determined using the DNS method. Since activity could now be detected for each autodisplay cellulase, the synergistic activity now had to be investigated. This was done by producing a mix from all three cellulases and determining the FP activity.

### FP Assay at Microtitre Plate Scale

Because of the total volume of 1.5 ml and the high quantities of DNS reagent (3 ml per mixture) to be used, the FPA according to IUPAC is very involved if one wants to investigate several cellulase mixes for FP activity. In the literature, the FP assay is described at microtitre plate scale with a total volume of 71 μl (Xiao Z et al, 2004). To do so, FP plates were produced with the aid of a punch. The ratio of reaction volume to filter paper was calculated via the filter paper area (FP plate vs. FP strip). This guaranteed that even at microtitre plate scale, the mixture was made in accordance with IUPAC. Since it was not known which is the best mixture ratio of the individual autodisplay cellulases, various mixtures were tested for FP activity. Mixtures without BglA strain were mixed with β-glucosidase from almonds. The host strain (BZ) and FP plate with buffer (BS) were used as controls. The FPase activity was determined after varying incubation periods at 60° C. via the reducing sugars released by means of DNS assay. Before the measurement, all mixtures were mixed for 10 min with β-glucosidase, to ensure that the cellobiose not yet converted is degraded into glucose, which results in a more precise determination of the reducing sugars. After separating the cells, the reducing sugars in the supernatant were determined at 540 nm. The result is shown in FIG. 20. Both the cellulase mix with commercially-available β-glucosidase and the mixture with all three autodisplay cellulases showed a conversion of the filter paper. After 5 days incubation at 60° C. the 50:50 (CelK:CeI5A) mix proved to be the best. The controls used were negative and showed no conversion of the filter paper (FIG. 20A). The quantity of reducing sugars released was determined in mg glucose/ml using the glucose standard curve (cf. FIG. 20B). After 5 days approx. 0.2 mg/ml was achieved. Because of the amorphous and crystalline structure of the cellulose, the degradation is not a linear reaction. In order to determine the FPase activity in FPU/I, therefore, as stated according to IUPAC, the 1 h measurement values were consulted. The result is summarised in Table 6.

TABLE 6

| CelK:Cel5A:BglA | FPU/I |
|---|---|
| 30:70:0 | 2.01 |
| 50:50:0 | 1.91 |
| 70:30:0 | 2.45 |
| 50:25:25 | 0.90 |

### FP-Assay Standard

Because of the low total volume (71 μl) used for the conversion of the FP plate, it proved to be difficult to separate the cells after 3 days, as the FP plate had absorbed almost all of the fluid. Therefore the standard FP assay according to IUPAC was conducted with a total volume of 1.5 ml. A mixture in microtitre plate scale was used as control. To do so, all three autodisplay cellulases were mixed in the same ratio and incubated, firstly with a 1×6 cm filter paper strip (standard) and secondly with a filter paper plate (micro) for 3 days at 60° C. The host strain and filter paper without cells (BS) were used as controls. The result is shown in FIG. 21. It can be seen that more reducing sugars are formed in the standard mixture after 3 days at 60° C., compared with the micro mixture. In the standard mixture the blending is better, as the cells are in contact at all times with the filter paper strip. In the micro mixture, a majority of the cells are above the filter paper plate, so not all cells are in contact with the FP at all times. The two controls used showed, as expected, no FPase activity (FIG. 21A). The quantity of reducing sugars released was determined with the aid of the glucose standard curve in mg glucose/ml (cf. FIG. 21B). In the micro mixture, after 3 days at 60° C., 0.06 mg/ml reducing sugars were determined. In contrast, in the standard mixture 0.38 mg/ml was achieved. Due to better blending, therefore, a 6.3 times better yield was achieved.

### Example 8

### Conversion of Poplar Wood

It was shown that the autodisplay cellulases can degrade filter paper (cf. FIG. 20). Since filter paper contains no lignin and only a very small proportion of hemicellulose, a more complex substrate would now have to be used. For this, steam pressure-treated poplar was used. The hemicellulose was removed by the pre-treatment, although lignin is still present. Lignin forms, firstly, a physical barrier, since it covers the surfaces of the cellulases, and secondly a chemical barrier, since cellulases can be bound by lignin and so, again, can no longer get to the cellulose. In order to investigate the poplar degradation of the cellulases, the autodisplay cellulases were mixed in equal parts and incubated with 4% poplar for 88 h at 60° C. The host strain and poplar in buffer without cells (BS) were used as controls. Following incubation, the poplar and the cells were sedimented by centrifugation. The supernatant was further used to determine reducing sugars formed by means of DNS assay. The result is shown in FIG. 22. The supernatants showed a yellowish colour even before measurement, similar to the DNS reagent. Therefore, after incubation with the DNS reagent, the absorption values at 540 nm were higher, compared with those from the FP assay, in which the supernatants are clear (cf. FIG. 20). It was not possible to determine the concentration in mg glucose/ml, since there were no adequate glucose standards available. Nevertheless, a higher absorption after 88 h at 60° C. was observed with the cellulases by comparison with the two controls used. The autodisplay cellulases are thus capable of converting even more complex substrates.

1. A nucleic acid molecule, comprising the following components:
(1) a section encoding a signal peptide,
(2) a section encoding a heterologous cellulase,
(3) an optional section encoding a protease recognition site,
(4) a section encoding a transmembrane linker, and
(5) a section encoding a transporter domain of an autotransporter or a variant thereof.

2. A nucleic acid molecule according to claim 1, characterised in that the cellulase is a beta-glucosidase or an endo- or exocellulase, preferably selected from the group comprising Bacillus subtilis endocellulase, Clostridium thermocellum exocellulase and Clostridium thermoceilum beta-glucosidase.

3. A nucleic acid molecule according to claim 1, characterised in that the transporter domain of an autotransporter is selected from the group comprising Ssp, Ssp-h1, Ssp-h2, PspA, PspB, Ssa1, SphB1, AspA/NaIP, VacA, AIDA-I, IcsA, MisL, TibA, Ag43, ShdA, AutA, Tsh, SepA, EspC, EspP, Pet, Pic, SigA, Sat, Vat, EpeA, EatA, EspI, EaaA, EaaC, Pertactin, BrkA, Tef, Vag8, PmpD, Pmp20, Pmp21, AgA1 protease, App, Hap, rOmpA, rOmpB, ApeE, EstA, Lip-1, McaP, BabA, SabA, AIpA, Aae, NanB and variants thereof.

4. A nucleic acid molecule according to claim 1, characterised in that it contains an expression control sequence operatively linked to the nucleic acid molecule, which can preferably be activated by adding isopropylthioglucopyranoside (IPTG) or arabinose.

5. A polypeptide, encoded by a nucleic acid molecule according to claim 1.

6. A micro-organism which expresses on its surface a polypeptide according to claim 5.

7. A micro-organism according to claim 6, characterised in that it is based on a gram-negative micro-organism.

8. A membrane fraction, obtainable from the cell according to claim 6.

9. A method for producing a reaction product using at least one cellulase, comprising the following steps:
(i) preparation of a micro-organism according to claim 6, and
(ii) bringing the micro-organism into contact with one or more cellulase substrates under conditions compatible with cellulase activity.

10. A method according to claim 9, characterised in that the product of the reaction catalysed by the cellulase is a mono-, di- or oligosaccharide, and that the at least one cellulase substrate is a polysaccharide source.

11. A method according to claim 9, characterised in that step (ii) is conducted at a pH in the range of 4.5 to 6.5.

12. A method according to claim 9, characterised in that step (ii) is conducted at a temperature in the range of 30 to 80° C.

13. A method according to claim 9, characterised in that in step (ii) a glucosidase is added.

14. A method according to claim 9, characterised in that it includes an additional step (iii) of recovering the micro-organism used in step (ii).

15. A method for producing a micro-organism, which presents a recombinant cellulase on its surface, including
a) the insertion of a nucleic acid sequence according to claim 1 in the micro-organism, and
b) optionally, the treatment of the micro-organism with a substance which activates the expression control sequence.

16. A micro-organism which has been transformed using a nucleic acid molecule according to claim 1.

17. The micro-organism according to claim 7, characterized in that it is based on Escherichia coli.

18. A method for producing a reaction product using at least one cellulase, comprising the following steps:
(i) preparation of a membrane fraction according to claim 8, and
(ii) bringing the membrane fraction into contact with one or more cellulase substrates under conditions compatible with cellulase activity.

19. A method according to claim 9, characterised in that step (ii) is conducted at a pH in the range of 5.5 to 6.5.

20. A method according to claim 9, characterised in that step (ii) is conducted at a temperature in the range of 50 to 65° C.
